(19) United States
(12) Patent Application Publication
(10) Pub. No.: US 2021/0315822 A1
(43) Pub. Date: Oct. 14, 2021

(54) BIOMIMETIC REBUILDING OF MULTIFUNCTIONAL RED BLOOD CELLS

(71) Applicants: Jimin Guo, Albuquerque, NM (US); C. Jeffrey Brinker, Albuquerque, NM (US); Jacob Ongudi Agola, Albuquerque, NM (US)

(72) Inventors: Jimin Guo, Albuquerque, NM (US); C. Jeffrey Brinker, Albuquerque, NM (US); Jacob Ongudi Agola, Albuquerque, NM (US)

(21) Appl. No.: 17/264,452

(22) PCT Filed: Jul. 30, 2019
(86) PCT No.: PCT/US2019/044107

§ 371 Date: Jan. 29, 2021

Related U.S. Application Data
(60) Provisional application No. 62/711,924, filed on Jul. 30, 2018.

(51) Int. Cl. A61K 9/127; C12N 5/078; A61K 35/18; A61K 47/69

(52) U.S. Cl. CPC A61K 9/1273; C12N 5/0641; C12N 2527/00; A61K 35/18; A61K 47/6913; A61K 9/1277

(57) ABSTRACT

Methods of preparing red blood cell mimetics and functionalized red blood cell mimetics, and methods of making and using those mimetics, are provided.

### CROSS-REFERENCE TO RELATED APPLICATIONS

This application claims the benefit of the filing date of U.S. application No. 62/711,924, filed on Jul. 30, 2018, the disclosure of which is incorporated by reference herein.

### STATEMENT OF GOVERNMENT SUPPORT

This invention was made with government support under DE-NA0003525 awarded by the United States Department of Energy/National Nuclear Security Administration. The government has certain rights in the invention.

### BACKGROUND

The integral functioning of cells remains a poorly understood subject because of the inherent complexity and fragility of biological systems (Sun et al., 2015a). To overcome these challenges, artificial cells are designed to simplify and mimic natural cell functions, as well as provide a platform to incorporate robust abiotic features resulting in so-called synthetic cells (Xu et al., 2016). This kind of design can be illustrated by the red blood cell (RBC) of higher organisms, which even though is simple in structure (e.g., lacking a cell nucleus and most intracellular organelles (Shang et al., 2014) is proving to be a suitable target for biomimicking and rebuilding of functional artificial cells. RBC possesses three main unique characteristics; special shape, flexibility/deformability, ability to carry oxygen, and long circulation times, that are the main biomimic focal points (Koshkaryev et al., 2013; Doshi et al., 2009). The biconcave discoidal shape of RBCs provides a favorable surface area-to-volume ratio and allows RBCs to undergo remarkable deformations while mechanical flexibility allows them to pass through restricted capillaries smaller than their own diameter (7 μm), a feature unmatched by typically stiff and spherical synthetic particles (Shang et al., 2014; Koshkaryev et al., 2013; Doshi et al., 2009). The hemoglobin contained in RBCs facilitates oxygen transport from the lungs to the body tissues through the formation of an oxyhemoglobin complex (Doshi et al., 2009). This function is aided by different molecular biomarkers on the RBC membrane that ensure self-recognition and evasion of the macrophages of the immune system, leading to long circulation times (Doshi et al., 2009; Su et al., 2016; Merkel et al., 2011).

There have been a few attempts to mimic the key structural and functional features of RBCs to generate artificial RBC replica materials. For example, Mitragotria, et al. (Doshi et al., 2009) developed synthetic biomaterial particles that closely mimic the shape, flexibility, and the ability to carry oxygen as natural red blood cells. Similarly, DeSimonea et al. (Merkel et al., 2011) explored the effect of mimicking RBC's shape and flexibility on the circulation time and biodistribution characteristics of an RBC-like material. While these studies presented encouraging findings, their main shortcoming is the predominant focus on one or two aspects of the RBC's unique characteristics, rather than mimicking a broad spectrum of RBC's properties that can lead to a potentially multifunctional RBC mimic construct. In addition, none of these previous studies ever considered the unique properties of the RBC membrane and how they impact long-term circulation times.

### SUMMARY

Red blood cells (RBCs) possess unique characteristics relative to other cells such as lack of cell nucleus and most intracellular organelles, deformable shape, oxygen carrying ability, and long circulation times. These features make RBC an attractive target for cellular biomimicking. As described herein below, a RBC mimic particle was rebuilt based on the unique characteristics of the native RBCs. In one embodiment, the RBC mimic particle was generated as follows; silica bio-replication of fixed RBCs, calcination of silicified RBC at 500° C. to remove organics and yield silica replicated RBCs, coating of the silica replicated RBCs with flexible polyion polymer template, buffered oxide etch (BOE) based desilicification, functionalization/loading of polymer replicated RBCs with different functional cargos and fusion with RBC membrane ghosts, to yield RBC mimic particle. The RBC mimic particle displayed a deformable property, zero hemolytic activity, and vascular flow in the ex ovo chick embryo vasculature. In addition, the RBC mimic particle is potentially loadable with various cargos including hemoglobin for oxygen transport, contrast agents for MRI imaging, and/or therapeutic small molecules, or combinations thereof. The RBC mimic particle can serve as a tool to advance the understanding of complex life processes and may be used as medicament delivery and/or bio-detection platform.

In one embodiment, a method to prepare a red blood cell mimetic is provided In one embodiment, vertebrate red blood cells are silified and calcinated, thereby providing silica replicated red blood cells; the silica replicated red blood cells are coated with more or more layers of one or more flexible polyion polymers; the coated silica replicated red blood cells are desilified; and the desilified, coated silica replicated red blood cells are fused with lipid bilayers, e.g., from vertebrate red blood cell ghosts (red blood cell membranes). In one embodiment, the vertebrate red blood cells that are silified and calcinated are fixed. In one embodiment, the desilified, coated silica replicated red blood cells are functionalized with one or more molecules. In one embodiment, the lipid bilayers are membranes from vertebrate red blood cell ghosts. In one embodiment, the red blood cells are from a mammal, e.g., a primate such as a human. In one embodiment, the silica replicated red blood cells are coated with two or more distinct polyionic polymers. In one embodiment, one polymer is a polycationic polymer and the other polymer is a polyanionic polymer. In one embodiment, the polyion comprises a polysaccharide, chitosan, polyarginine, or polylysine. In one embodiment, the polyion comprises alginate, pectin, xanthan gum, carboxymethyl cellulose, dextran sulfate, agar, or carrageenan. In one embodiment, the polyion comprises a polysaccharide, chitosan, chitin, polyarginine, polylysine, polyacrylamide, or poly(N-isopropylacrylamide). In one embodiment, the polyion comprises alginate, pectin, xanthan gum, carboxymethyl cellulose, dextran sulfate, agar, carrageenan, hyaluronate, polylactic acid, poly(lactic-co-glycolic acid), polyacrylate, or polymethacrylic acid. In one embodiment, the cells are fixed with formaldehyde, acrolein, glyoxal, osmium tetroxide, carbodiimide, diimidoester, chloro-s-traizide, diisocyanate, ethanol, picric acid, or glutaraldehyde. In one embodiment, the one or more molecules comprise nucleic acid, protein, peptides, antibodies, or drugs. e.g., siRNA, hemoglobin, insulin, heparin, secretin, bivalirudin, vasopressin, pramlintide, corticotropin, bacitracin, teriparatide, chemotherapeutics, an anti-cancer agent, an anti-viral agent, an anti-bacterial agent, or an anti-parasite agent, e.g., doxorubicin, afatinib, rapamycin, interferon, acyclovir, ribavirin, zidovudine, penicillin, vancomycin, erythromycin, cephalosporin, rifampin, albendazole, or nitazoxanide, or hemoglobin. In one embodiment, the one or more molecules comprise a cytokine or growth factor, e.g., e.g., adrenomedullin, angiopoietin, bone morphogenetic proteins, leukemia inhibitory factor, macrophage colony-stimulating factor, granulocyte colony stimulating factor, epidermal growth factor, ephrin, erythropoietin, hepatocyte growth factor, insulin, interleukin, myostatin, platelet-derived growth factor, tumor necrosis factor-alpha, vascular endothelial growth factor. In one embodiment, the one or more molecules comprise a contrast agent. In one embodiment, the desilifying comprises etching. In one embodiment, the red blood cell mimetic comprises a single layer of one polyion polymer. In one embodiment, the red blood cell mimetic comprises two, three four or more layers or polyion polymer including alternating layers of different polyion polymers. In one embodiment, the vertebrate is a mammal. In one embodiment, the mammal is a primate, e.g., a human, a bovine, equine, canine, feline, ovine, swine, or caprine. In one embodiment, the mimetic has a discoid shape, a flexible discoid shape. In one embodiment, the mimetic has a biconcave discoid shape, a flexible biconcave discoid shape.

Further provided are functionalized red blood cell mimetics prepared by the method. In one embodiment, the one or more molecules are between the outermost polyion layer(s) and the lipid bilayer. In one embodiment, the one or more molecules are between one or more of the polyion layers. In one embodiment, the one or more molecules are within the innermost polyion layer. In one embodiment, the red blood cell mimetics further comprise functionalizing the lipid bilayer, e.g., synthetic lipid may be added into the lipid bilayer to adjust the mobility of lipid bilayer, and a targeting ligand may be added on the outer layer to generate targeting properties. In one embodiment, the lipid bilayer is functionalized before fusion. In one embodiment, the lipid bilayer is functionalized after fusion. In one embodiment, the mimetic has a discoid shape, a flexible discoid shape. In one embodiment, the mimetic has a biconcave discoid shape, a flexible biconcave discoid shape.

### BRIEF DESCRIPTION OF THE FIGURES

FIGS. 1A-1B. A) Preparation of RBC mimic particle via silicification of RBC, calcination, polyion polymer coating, crosslinking, silica etching, and RBC membrane ghost fusion. B) Depiction of the structure and properties of functional RBC mimic particle: polymer core provides biconcave shape and mechanical flexibility properties, the functional cargo core provides cargo delivery and bio-detection capabilities, and membrane coating provides self-antigens and immune-evasive properties.

FIGS. 2A-2C. A) Schematic of synthetic RBCs preparation, B) Zeta potential of the engineering steps of synthetic RBCs, C) Confocal microscopy and differential interference contrast (DIC) images of RBC mimic particle. RBC-shaped polymer core (green) was prepared by FITC-modified chitosan and RBC-membrane-derived ghost (red), which was mixed with 1,2-dioleoyl-sn-glycero-3-phosphoethanolamine-N-(lissamine rhodanine B sulfonyl) (18:1 Liss Rhod PE) lipid, and fused onto the polymer core. The inset images are of higher magnification of RBC mimic particle.

FIGS. 3A-3F. A) Schematics of the blood capillaries and microfluidic blood capillary model, B) Illustration of the available flow paths for RBC mimic particles and control particles in the microfluidic blood capillary model, overlaid on the bright field image of microfluidic blood capillary model, C) Bright field image of RBC mimic particle at the outlets of the microfluidic device after passing through the small dimension (5 μm) capillaries Note that the RBC mimic particle were not visibly damaged by the deformation. D) Trajectory analysis of RBC mimic particles (cross-linker concentration of about 4%, 8%, and 12%) and controls (silica RBC replicas and RBCs). This analysis was done by dividing the number of particles passing through the capillaries by the number of particles directly through the sample-side. Mean±standard deviation, n=3. E,F) Time-lapse fluorescence microscopy images of RBC particles (gray) stopping (cross-linker concentration=12%) or passing through (cross-linker concentration about 4%) the microfluidic device capillaries. The pressure drop across the capillaries is about 6 mbar in E) and F). The “drawn out” shape of the RBC mimic particles observed in some of the time-lapse frames is due to the acquisition rate of the imaging camera. The biconcave shape is again observed when the RBC mimic particles are no longer in motion (as seen in C).

FIGS. 4A-4C. A) Blood types, antigen present on the RBC, and corresponding antibodies-mediated agglutination. B) Illustration of antibodies-mediated agglutination. C) The bright field images of human type A⁺ RBCs (RBC-A⁺) and RBC mimic particles with human type A⁺ RBC membrane ghosts (RRBC-A⁺) incubated with different anti-typing sera for 30 minutes. Scale bar=25 μm.

FIGS. 5A-5D. A) Percentage of lysed human red blood cells after exposure (for 2 hours at 37° C.) to 8×10⁶ and 4×10⁷ particles/mL of different RBC shaped particles (silica RBC replica[a], silica RBC replica@polymer[b], RBC polymer replica[c], silica Synthetic RBC [d], and synthetic RBC[e]). Mean±standard deviation, n=3. B) HUVEC cell viability after incubated with different RBC shaped particles, C) Raw 264.7 cell viability after incubated with different RBC shaped particles.

FIGS. 6A-6C. Fluorescence microscopy image of: RBC polymers, silica synthetic RBCs, and RBC mimic particle within the chicken embryo deep blood vessel (top) and capillary bed (bottom). The bottom inset images are higher magnification images of RBC polymers, silica synthetic RBCs, and RBC mimic particle within the chicken embryo capillary bed.

FIGS. 7A-7D. A) Visual confirmation of oxygen carrying capacity by luminol reaction. B) UV-Vis spectrum analysis of a oxygenated and deoxygenated RBC mimics, C) Oxygenation and deoxygenation of RBC mimics, D) Oxygenation curve for native and RBC mimics.

FIGS. 8A-8E. A) Drug release percentage for Mn-TPPS4-loaded RBC mimic maintained in extracellular physiological conditions (PBS, pH 7.4) at 37±° C., B) Drug release percentage for DOX-loaded RBC mimics maintained in extracellular physiological conditions (PBS, pH 7.4) at 37° C., C) Photographs of a dispersion of magnetic RBC mimics before (left) and after (right) placing a magnet on its side, D) Higher magnification bright field microscopy images of magnetic r RBC mimics, that magnetic RBC mimics maintain the biconcave shape of native RBCs, E) Bright field microscopy images of magnetically-moved magnetic RBC mimics.

FIGS. 9A-9C A) Scanning Electron Microscopy (SEM) images of fixed RBC, silica RBC replica, silica RBC replica with polymer, and RBC polymer replica. B) Zeta potential (a changes during layer by layer polymer assembly, C) SEM images of silica RBC replica@polymer during the silica etching process to form RBC polymer replica. Diluted buffered oxide etch (1:10 diluted BOE) was used to etch the silica part from the silica RBC replica@polymer.

FIG. 10. Si measurement by ICP-MS of silica RBC replica@polymer during the silica etching process to form RBC polymer replica. Diluted buffered oxide etch (1:10 diluted BOE) was used to etch the silica part from the silica RBC replica@polymer.

FIG. 11. The bright field images of human type B⁺ RBCs (RBC-13+) and RBC mimic particles with human type B⁺ RBC membrane ghosts (RRBC-B⁺) incubated with different anti-typing sera for 30 minutes. Scale bar=25 μm.

FIG. 12. The fluorescence and bright field microscopy images of RBCs and RBC mimic particles stained with anti-ICAM-4 and anti-CD47 antibodies. Scale bar=25 μm.

FIGS. 13A-13B. Schematic illustration of the design and construction of rebuilt RBC via silicification of RBC, calcination, polyion polymer coating, crosslinking, silica etching, and RBC membrane ghost fusion (A) and the properties of rebuilt RBC (B): circulation and oxygen delivery (a), cargo delivery (b), and detoxification and toxin sensing(c).

FIGS. 14A-14H. A) Scanning Electron Microscopy (SEM) images of silica RBC replica with polymer. Scale bars, 2 μm. B) Fluorescence image of RBC-membrane-derived ghost. Scale bars, 10 μm. C) Differential interference contrast (DIC) images of rebuilt RBC. Scale bars, 2 μm. D) DIC images of rebuilt RBC. Scale bars, 10 μm. E) Zeta potential of the synthetic steps to create rebuilt RBC. The zeta potential measurements for RBC* and RRBC* was acquired in 154 mM NaCl solution, the rest were measured in 5 mM NaCl solution. F-H) Confocal microscopy images of rebuilt RBC. Scale bars, 20 μm. The inset images are of higher magnification of rebuilt RBC. Scale bars, 3 μm.

FIGS. 15A-15G. Schematics of the blood capillaries (A) and microfluidic blood capillary model (B). C) Illustration of the available flow paths for rebuilt RBC and control particles in the microfluidic blood capillary model, overlaid on the bright field image of microfluidic blood capillary model. Scale bars, 100 μm. D) Bright field image of rebuilt RBC at the outlets of the microfluidic device after passing through the small dimension (5 μm) capillaries. Scale bars, 5 μm. Note that the rebuilt RBC were not visibly damaged by the deformation. E) Trajectory analysis of rebuilt RBC (cross-linker concentration of about 4%, 8%, and 12%) and controls (silica RBC replicas and RBCs). This analysis was done by dividing the number of particles passing through the capillaries by the number of particles directly through the arterial side. Mean±standard deviation, n=3. F, G) Time-lapse fluorescence microscopy images of rebuilt RBC (grey) stopping (cross-linker concentration about 12%, F) or passing through (cross-linker concentration about 4%, G) the microfluidic device capillaries. Scale bars, 5 μm. The pressure drop across the capillaries is about 6 mbar in F and G. The “drawn out” shape of the rebuilt RBC observed in some of the time-lapse frames is due to the acquisition rate of the imaging camera. The biconcave shape is again observed when the rebuilt RBC are no longer in motion (as seen in C).

FIGS. 16A-161. A-C) Optical images of chicken embryo. Scale bars, 10 mm (A), 3 mm (B), and 1 mm (C). D-I) Fluorescence microscopy image of: RBC polymer replica, silica rebuilt RBC, and rebuilt RBC within the chicken embryo CAM deep blood vessel (D-F) and capillary bed (G-I). The bottom inset images are higher magnification images of RBC polymer replica, silica rebuilt RBC, and rebuilt RBC within the chicken embryo capillary bed. Scale bars, 50 μm (D-I) and 5 μm (inset images of G-I).

FIGS. 17A-17G. A) Circulation time of rebuilt RBCs (n=3; mean±SD). Insert table is the related elimination half-life. B) Fluorescence intensity per gram of tissue at 2, 6, 24 and 48 h after intravenous administration of rebuilt RBC (n=3; mean±SD). C) Relative signal per organ at 2, 6, 24 and 48 hours after intravenous administration of rebuilt RBC (n=3; mean±SD). D) UV-Vis spectrum analysis of oxygenated and deoxygenated rebuilt RBC. Insert images show the generation of bluish glow of native and rebuilt RBCs after the addition of luminol-perborate mixture. E) Schematic illustration of the oxygen binding via hemoglobin by RBCs. F) Reversible transfer of rebuilt RBC between oxygenation and deoxygenation states. G) Time-dependent oxygenation curves of native and rebuilt RBC.

FIGS. 18A-18K. A) Schematic illustration of the cargo release. B) Schematic illustration of multifunctional rebuilt RBC delivery drug in tumor area. C) Schematic illustration of the manipulation of magnetic rebuilt RBC via an external magnetic field. D) Release percentage for Mn-TPPS4-loaded rebuilt RBC maintained in extracellular physiological conditions (PBS, pH 7.4) at 37° C. E) Drug release percentage for DOX-loaded rebuilt RBC maintained at 37° C. F,G) Photographs of a dispersion of magnetic RBC mimics before (F) and after (G) placing a magnet on its side. H) Bright field microscopy images of magnetically-moved magnetic rebuilt RBC. Scale bars, 10 μm. I) Schematic illustration of detoxification and toxin sensing property of rebuilt RBC. J) Hemolysis analysis of samples with α-hemolysin. K) Luminescence intensity of biosensor loaded RBC polymer replica and rebuilt RBC under different conditions.

FIGS. 19A-19C. A) Scanning Electron Microscopy (SEM) images of fixed RBC, silica RBC replica, silica RBC replica with polymer, and RBC polymer replica. Scale bar=2 μm. B) Zeta potential (0 changes during layer by layer polymer assembly, C) SEM images of silica RBC replica@polymer during the silica etching process to form RBC-polymer replica. Diluted buffered oxide etch (1:10 diluted BOE) was used to etch the silica part from the silica RBC replica@polymer. Scale bar=5 μm.

FIG. 20. Si measurement by ICP-MS of silica RBC replica©polymer during the silica etching process to form RBC-polymer replica. Diluted buffered oxide etch (1:10 diluted BOE) was used to etch the silica part from the silica RBC replica@polymer.

FIGS. 21A-21C. A) Blood types, antigen present on the RBC, and corresponding antibodies-mediated agglutination. B) Illustration of antibodies-mediated agglutination. C) The bright field images of human type A⁺ RBCs (RBC-A⁺) and RRBC particles with human type A⁺ RBC membrane ghosts (RRBC-A⁺) incubated with different anti-typing sera for 30 minutes. Scale bar=25 μm.

FIG. 22. The bright field images of human type B⁺ RBCs (RBC-B⁺) and RRBC particles with human type B⁺ RBC membrane ghosts (RRBC-B⁺) incubated with different anti-typing sera for 30 minutes. Scale bar=25 μm.

FIG. 23. The fluorescence and bright field microscopy images of RBCs and RRBC particles stained with anti-ICAM-4 and anti-CD47 antibodies. Scale bar=25 μm.

FIGS. 24A-24D. A) Percentage of lysed human red blood cells after exposure (for 2 h at 37° C.) to 8×10⁶ and 4×10⁷ particles/mL of different RBC shaped particles (silica RBC replica[a], silica RBC replica@polymer[b], RBC-polymer replica[c], silica RBC replica@polymer-RBC ghost (silica-RRBC) [d], and RRBC[e]). Mean±standard deviation, n=3. B) HUVEC cell viability after incubated with different RBC shaped particles, C) Raw 264.7 cell viability after incubated with different RBC shaped particles.

FIG. 25. Hemolysis activity of α-hemolysin alone or preincubated with RRBC particles.

FIG. 26. Luminescence of RBC in different hemolysis content after incubation with biosensor loaded RRBC particles.

### DETAILED DESCRIPTION

A modular design approach was employed to rebuild an artificial RBC or RBC mimic particle which can fully mimic broad properties of native RBCs as well as perform additional abiotic functions not inherent to the native RBCs, e.g., delivery of molecules including but not limited to drugs, antibodies, nanoparticles, imaging agents, or biosensors, e.g., calcein, mCB, SP2, substrate for a protease, e.g., labeled substrates, such as a substrate for throbin.

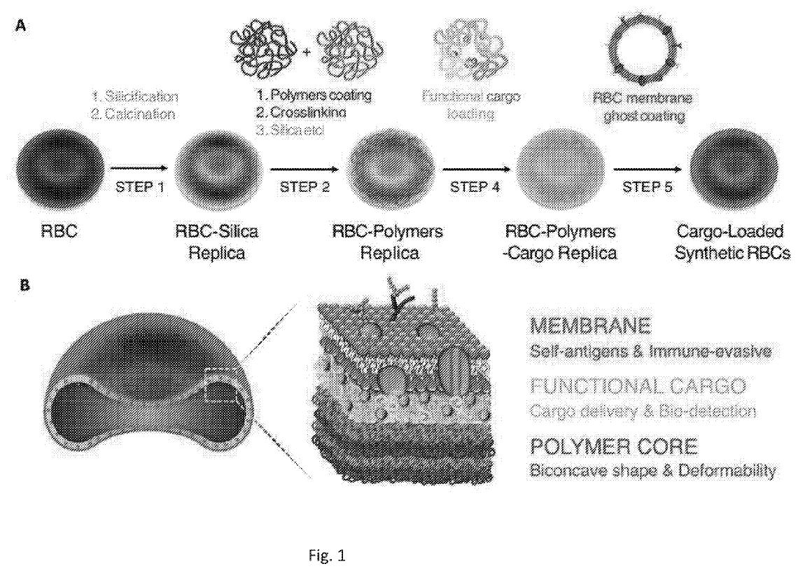

In a modular design, a system is subdivided into smaller and independently created modules, which are then linked together by well-defined interfaces to generate a functional unit (Rollie et al., 2012). The standardization of individual modules enables a reliable prediction and optimization of a system's behavior. Based on this strategy, RBC mimic particles were prepared using three separate building blocks (FIG. 1); silica cell bio-replication, a layer-by-layer self-assembly process to translate native RBCs into a flexible RBC-shaped polymer core loadable with functional cargos (e.g., hemoglobin) to provide special functions, and encapsulation with RBC derived membrane ghosts mimicking the membrane surface properties of the native RBCs. The final artificial RBC system provides a better understanding of the relationship between structure on the one hand and the functions of the native RBCs, helping us design and build an artificial cell which can serve as a great tool to advance our understanding of living systems.

### EXEMPLARY EMBODIMENTS

In one embodiment, functionalized red blood cell mimetics are provided. In one embodiment, one or more molecules are between the polyion layer and the lipid bilayer of the mimetic. In one embodiment,

one or more molecules are between polyion layers of the mimetic. In one embodiment, the lipid bilayer of the mimetic is functionalized. In one embodiment, a red blood cell mimetic comprising a lipid bilayer surrounding a discoid formed of etched silica and at least one polyion layer is provided. In one embodiment, the mimetic further comprises at least one diagnostic or therapeutic molecule. In one embodiment, the mimetic comprises a contrast agent. In one embodiment, the mimetic further comprises at least one molecule that is a substrate for an enzyme. In one embodiment, the mimetic further comprises at least one molecule that is a chemotherapeutic agent. In one embodiment, the mimetic of further comprises at least one molecule that is a nanoparticle. In one embodiment, the lipid bilayer of the mimetic is from vertebrate red blood cell membranes. In one embodiment, the mimetic comprises two or more distinct polyionic polymers. In one embodiment, the mimetic comprises alternating layers of two or more distinct polyionic polymers. In one embodiment, one polymer is a polycationic polymer and the other polymer is a polyanionic polymer. In one embodiment, the polyion comprises a polysaccharide, chitosan, polyarginine, or polylysine. In one embodiment, the polyion comprises alginate, pectin, xanthan gum, carboxymethyl cellulose, dextran sulfate, agar, or carrageenan. In one embodiment, the mimetic further comprises isolated nucleic acid, protein, peptides, antibodies, drugs, or hemoglobin. In one embodiment, the mimetic further comprises an anti-cancer agent, an anti-viral agent, an anti-bacterial agent, or an anti-parasite agent. In one embodiment, the mimetic further comprises a cytokine or growth factor.

In one embodiment, a method to prepare a red blood cell mimetic is provided. In one embodiment, the method includes coating silica replicated vertebrate red blood cells with one or more polyion polymers in one or more layers, thereby yielding coated silica replicates red blood cells; etching the coated silica replicated red blood cells, thereby yielding red blood cell-polymer replicas; and fusing the red blood cell-polymer replicas with lipid bilayers. In one embodiment, the method includes etching polyion polymer coated silica replicated vertebrate red blood cells, thereby yielding red blood cell-polymer replicas; and fusing the red blood cell-polymer replicas with lipid bilayers. In one embodiment, the method includes silificating and calcinating vertebrate red blood cells, thereby providing silica replicated vertebrate red blood cells; coating the silica replicated vertebrate red blood cells with one or more polyion polymers in one or more layers, thereby yielding coated silica replicates red blood cells; etching the coated silica replicated red blood cells, thereby yielding red blood cell-polymer replicas; and

fusing the red blood cell-polymer replicas with lipid bilayers. In one embodiment, prior to silification the vertebrate red blood cells are fixed. In one embodiment, the red blood cells are non-primate
mammalian or primate cells. In one embodiment, the cells are human red blood cells. In one embodiment,
the desilified, coated silica replicated red blood cells are functionalized with one or more molecules. In one embodiment, at least one of the molecules is a contrast agent. In one embodiment, at least one of the molecules is a substrate for an enzyme. In one embodiment, at least one of the molecules is a chemotherapeutic agent. In one embodiment, at least one of the molecules is a nanoparticle. In one embodiment, the lipid bilayers are membranes from vertebrate red blood cells. In one embodiment, the silica replicated red blood cells are coated with two or more distinct polyionic polymers. In one embodiment, the coat has alternating layers of the two or more distinct polyionic polymers. In one embodiment, one polymer is a polycationic polymer and the other polymer is a polyanionic polymer. In one embodiment, the polyion comprises a polysaccharide, chitosan, polyarginine, or polylysine. In one embodiment, the polyion comprises alginate, pectin, xanthan gum, carboxymethyl cellulose, dextran sulfate, agar, or carrageenan. In one embodiment, the cells are fixed with formaldehyde, acrolein, glyoxal, osmium tetroxide, carbodiimide, diimidoester, chloro-s-traizide, diisocyanate, ethanol, picric acid, or glutaraldehyde. In one embodiment, the one or more molecules comprise nucleic acid, protein, peptides, antibodies, drugs, or hemoglobin. In one embodiment, the one or more molecules comprise an anti-cancer agent, an anti-viral agent, an anti-bacterial agent, or an anti-parasite agent. In one embodiment,
the one or more molecules comprise a cytokine or growth factor. In one embodiment, the one or more molecules comprise a MRI contrast agent.

Use of a population of red blood cell mimetics comprising a lipid bilayer surrounding a discoid formed of etched silica and at least one polyion layer is also provided, e.g., in biosensing, imaging or drug delivery.

In one embodiment, a method is providing that includes administering to a mammal a composition

comprising a population of the mimetic. In one embodiment, the mammal is a human. In one embodiment,
the mimetic comprises one or more molecules, e.g., isolated protein, isolated nucleic acid, or an antibody or an antigen binding portion thereof. In one embodiment, the lipid bilayer comprises one or more moieties, e.g., a protein ligand such as an antibody or antigen binding portion thereof. In one embodiment,
the composition is systemically administered. In one embodiment, the composition is locally administered.

The invention will be described by the following non-limiting examples.

### Example 1

Materials and Methods

Materials

All chemicals and reagents were used as received. Tetramethyl orthosilicate 99%, TMOS), sodium chloride (NaCl), hydrochloric acid (37%, HCl), chitosan, alginate, formaldehyde (37%), and glutaraldehyde solution (25% in H₂O) were purchased from Sigma-Aldrich. 1×phosphate-buffered saline (1×PBS) was purchased from Thermo

Scientific. Buffered Oxide Etch (BOE) was purchased from KMG Chemicals while 1,2-dioleoyl-sn-glycero-3-phosphoethanolamine-N-(lissamine rhodamine B sulfonyl) (18:1 Liss Rhod PE) lipid was purchased from Avanti Lipids. Ethanol was purchased from KOPTEC. Human umbilical vein endothelium cells (HUVEC) and mouse macrophage Raw264.7 cells were obtained from the American Type Culture Collection (ATCC).

Purification of RBCs

Human RBCs were acquired from healthy donors with their informed consent. All blood samples were collected and stored in BD Vacutainer® blood collection tubes (Becton Dickinson, N.J., USA) containing 1.5 mg of EDTA per mL of blood for anticoagulation purposes. The purification of whole blood was carried out using Ficoll® density gradient centrifugation procedure.

Preparation of Silica RBC Replicas

Purified RBCs were fixed in 4% formaldehyde in 1×PBS at room temperature for 20 hours before silicification. The fixed RBCs were rinsed twice with 1×PBS, once with 154 mM NaCl solution (0.9% saline) and then suspended in a silicification solution containing 100 mM TMOS, 154 mM NaCl and 1.0 mM HCl (pH 3.0). After 24 hours rotation at room temperature to allow silicification process to take place, silicified RBCs were subjected to series of ethanol dehydration (30, 50, 70, 90, 100% ethanol in water) for 10 minutes each and then dried under vacuum for 24 hours. Dry silicified RBCs were then calcined at 500° C. for 4 hours in an oven by placing them in a covered (but not air tight) glass tube to generate silica RBC replicas.

Preparation of RBC-Shaped Polymer Cores

The silica RBC replicas were incubated for 2 hours in chitosan solution (2 mg/mL in 1% acetic acid solution) under constant shaking. After rinsing with water, the particles were resuspended in alginate solution (1 mg/mL in water) under constant shaking for 0.5 hours. Then, the particles were rinsed with water and isolated via centrifugation (1500 g for 5 minutes). This process represents the typical procedure for single chitosan-alginate layer formation and it was repeated two times to achieve polymer coated silica RBC replica. In order to fabricate RBC-shaped polymer core, 1:10 diluted buffered oxide etch, also known as buffered HF (BOE) solution (pH about 5) was used to etch the silica and yield RBC-shaped polymer cores. The RBC-shaped polymer cores were washed with water and resuspended in double distilled water.

Preparation of RBC-Membrane-Derived Ghosts

Purified RBCs were washed three times with ice cold 1×PBS, and then suspended in ice cold 0.25×PBS for 20 minutes to allow hemolysis to take place. After treatment with hypotonic solution (0.25×PBS), the released hemoglobin was removed via centrifugation (1000 g for 5 minutes), whereas the pellet (RBC ghost) with light pink color was collected and washed twice with 1×PBS. The RBC ghosts (devoid of cytoplasmic contents) were mixed with 1,2-dioleoyl-sn-glycero-3-phosphoethanolamine-N-(lissamine rhodamine B sulfonyl) (18:1 Liss Rhod PE) and verified under fluorescence microscope, which revealed a hollow spherical structure of RBC membrane ghosts.

Preparation of RBC Mimic Particles

To prepare the RBC mimic particles, the RBC membrane ghosts were fused onto RBC-shaped polymer core. Briefly, 4×10⁷ RBC-shaped polymer cores quantified by hemocytometer were mixed with RBC membrane ghosts prepared from 1×10⁸ purified RBCs and then sonicated in ice cold water bath for 1 minute. An excess of RBCs was used to compensate for the membrane loss during RBC ghost derivation. The resulting RBC mimic particles were carefully centrifuged (5000 g for 10 minutes) and the excess membrane components remaining in the supernatant were removed.

Zeta Potential Measurements

Zeta potential measurements were made using Malvern Zetasizer Nano-ZS (Westborough, Mass., USA) equipped with a He—Ne laser (633 nm) and non-invasive backscatter optics (NIBS). The layer-by-layer samples, silica RBC replica, RBC-shaped polymer core, and RBC mimic particles for zeta potential measurements were suspended in water, while the zeta potential measurements for the RBC was acquired in 154 mM NaCl solution (0.9% saline) using monomodal analysis tool. All reported values correspond to the average of at least three independent samples.

Scanning Electron Microscopy (SEM) Imaging

The morphology of fixed RBC, silica RBC replica, polymer coated silica RBC replica and RBC-shaped polymer core samples were characterized using scanning electron microscope (SEM). SEM samples were prepared by drop casting. Briefly, all samples were suspended in water, and then dropped onto 5×5 mm glass slides. The glass slides were then mounted on SEM stubs using conductive adhesive tape (12 mm OD PELCO Tabs). Samples were sputter coated with a 10 nm layer of gold using a Plasma Sciences CrC-150 Sputtering System (Torr International, Inc.). SEM images were acquired under high vacuum, at 10 kV, using an FEI Quanta series scanning electron microscope (Thermo Fisher Scientific, MA, USA).

Confocal Microscopy Imaging

RBC mimic particles were spotted onto glass cover slips. Slides were mounted using Vectashield Antifade. Confocal images were acquired with a 63×/1.4NA oil objective in sequential scanning mode using a Leica TCS SP8 confocal microscope.

Microfluidic Blood Capillary Model and Experiments

The microfluidic blood capillary model mimicking dimensions (5 μm in diameter and 50 μm in length) and pressure drops of human blood capillaries was prepared as described previously (Kozlovskaya et al., 2014). In brief, a master pattern was designed using computer-aided design software (AutoCAD 2013, Autodesk, USA) and then simulation software (COMSOL Multiphysics 4.3, USA) was used to refine and validate the design. The designed master pattern was then transferred to the silicon wafer using chrome mask and negative photoresist and then transferred into polydimethylsiloxane (PDMS) to achieve microfluidic blood capillary device through soft lithography. The microfluidic experiments were then performed as follows. The non-sample reservoirs were filled with 1×PBS, and the device was then connected to a pressure controller (NE-300, New Era Pump System) and placed on top of a Zeiss AxioExaminer upright microscope. For the microfluidic tests, 1×10⁶/mL particles (RBCs, silica RBC replicas, and RBC mimic particles cross-linked with different cross-linker concentrations) in PBS were analyzed. The different pressures used for the two inlets were chosen based on the finite element simulations to achieve physiologically relevant pressure drops over the microchannels (Table 1). Videos of particle trajectories were recorded for each pressure differential and with each sample.

TABLE 1

| Simulation of flow in the | | |
|---|---|---|
| microfluidic blood capillary | | |
| model. Average pressure | Flow rate at | Flow rate at |
| drop across central | high pressure | low pressure |
| microchannels, mbar | outlet, μL min-1 | outlet, μL min-1 |
| 2.2 | 18 | 10 |
| 6.7 | 34 | 12 |
| 20 | 79 | 16 |

Antibody-Mediated Agglutination Assay

Briefly, 1×10⁶ native RBCs or RBC mimic particles were suspended in 450 μL of 1×PBS (pH 7.4) solution, and then 50 μL of anti-type sera [anti-A, anti-B, and anti-D (Rh)] were added. After 15 minutes, the bright field images were acquired on the Leica DM13000 B inverted microscope to evaluate occurrence of agglutination or lack thereof.

Immunofluorescence Staining

The native RBC and RBC mimic particles were blocked with 5% BSA in 1×PBS, and then incubated with fluorescent antibodies against ICAM-4 (R&D Systems) and CD47 (Biolegend) proteins for 30 minutes. The samples were then rinsed with 1×PBS, and then suspended again in 1×PBS. Microscopy images were then obtained on the Leica DM13000 B inverted fluorescence microscope.

Hemolysis Assay

Purified RBCs were incubated with different concentrations of silica RBC replicas, polymer coated silica RBC replicas, RBC-shaped polymer cores, and RBC mimic particles at 37° C. for 2 hours in continuous rotating state. Double distilled (D.I.) water and 1×PBS containing purified RBCs were used as the positive and negative controls, respectively. The absorbance of hemoglobin in the supernatant was measured by a BioTek microplate reader (Winooski, Vt.) at 540 nm. The hemolysis percentage of each sample was determined using the reported equation (Sun et al., 2015b; Cohen et al., 2009) as; Percent hemolysis (%)=100*(Sample Abs_{540 nm}−Negative control Abs_{540 nm})/(Positive control Abs_{540 nm}−Negative control Abs_{540 nm})

Cell Viability Assay

Cell culture was performed using standard procedures. HUVEC and Raw264.7 cells were maintained in the respective media of endothelial cell growth medium and DMEM containing 10% FBS at 37° C. and 5% CO₂. Cells were passaged at approximately 70% confluency. For cell viability assays, 100 μL of cell suspension (100,000 cells/mL) were seeded into a 96-well plate (White Opaque) and cultured for 24 hours at 37° C. The cells were then incubated with 100 μL of different concentrations of silica RBC replicas, polymer coated silica RBC replicas, RBC-shaped polymer cores, and RBC mimic particles solutions. After 24 hours incubation, 100 μL of CellTiter-Glo 2.0 Reagent was added into each well and incubated for 10 minutes at room temperature. The luminescence readings were then obtained/recorded using BioTek microplate reader. The percent cell viability was calculated relative to the control non-treated cells.

Test of Vascular Flow in Ex Ovo Chick Embryos

The vascular flow characteristics of RBC mimic particles were tested using Ex ovo chick embryo model as described previously (Sun et al., 2015b) and was conducted following institutional approval (Protocol 11-100652-T-HSC). Briefly, eggs were acquired from East Mountain Hatchery (Edgewood, N. Mex.) and placed in a GQF 1500 Digital Professional incubator (Savannah, Ga.) for 3-4 days. Embryos were then removed from shells by cracking into 100 ml polystyrene weigh boats. Ex ovo chick embryos were covered and incubated at 37° C., 70% humidity. 50 μL (at 4×10⁷ particles/mL) of samples (RBC-shaped polymer cores, silica RBC replica@polymer-RBC ghost, and RBC mimic particles) in 1×PBS were injected into the secondary or tertiary veins via pulled glass capillary needles and then, the CAM vasculature was imaged using a customized avian embryo chamber and a Zeiss Axio Examiner upright microscope with heated stage.

Hemoglobin Loading

The chitosan surface of the RBC-polymer core was incubated for 24 hours with 5 mg/mL hemoglobin under constant shaking at 4° C. After rinsing with water, the particles were resuspended in 2 mg/mL chitosan solution under constant shaking for 0.5 hours at 4° C. This process was repeated three times to achieve enough hemoglobin loading. The particles were then resuspended in 1 mg/mL alginate solution under constant shaking for 0.5 hours and then the RBC membrane ghosts were fused onto hemoglobin loaded RBC-shaped polymer core to generate hemoglobin loaded RBC mimic.

Chemiluminescence Assays

The luminol-based method for evaluating oxygen carrying ability of the RBC mimic was adopted from Doshi et al. (2009). Briefly, 70 mg sodium perborate, 500 mg sodium carbonate, and 200 mg luminol were added to 5 mL water and dissolved with sonication to achieve luminol solution. The luminol solution was left undisturbed for 5 minutes in a dark room. For imaging purposes, 1 mL of luminol solution was added to 4 mL samples (5 million native RBCs and RBC mimic particles) in 1×PBS (pH 7.4) solution. The optical image was taken by Sony ILCE-5100 Camera (ISO-100 and exposure time 1/15 seconds). The chemiluminescence optical image was taken in a dark room by Sony ILCE-5100 Camera (ISO-6400 and exposure time 30 seconds). For luminescence assay, 100 μL of samples (5 million native RBCs and RBC mimic particles) in 1×PBS (pH 7.4) solution were added into white 96-well plates at a density of 5 million cells/mL. After that, 20 μL of luminol solution was added to each well. Mix the contents for 2 minutes on shaker in the dark. Luminescence was measured using a BioTek microplate reader. The luminescence was expressed as a relative percentage of the control.

Assay of the Reversible Binding of Oxygen by RBC Mimic

The ability of the RBC mimic to reversibly binding oxygen was detected by analyzing changes of UV-Vis absorption spectrum (300-700 nm) in oxygenated and deoxygenated solutions (Jia et al., 2012; Duan et al., 2012). For complete deoxygenation, nitrogen gas was bubbled into the sample solution to displace oxygen. After 2 hours, sodium dithionite (Na₂S₂O₄) was added, and UV-Vis absorption spectrum was obtained by a BioTek microplate reader. For oxygenation, sample solutions were exposed to atmospheric oxygen for more than 2 hours, and then UV-Vis absorption spectrum was recorded as before. This process represents the typical procedure used to test reversible oxygen binding capability and it was repeated two times. The deoxygenated sample (λₘₐₓ=430 nm) could be gradually converted to oxygenated sample (λₘₐₓ=415 nm) by exposing it to air atmosphere at room temperature. The oxygenation rate of the deoxygenated sample was monitored by observing changes in absorbance via UV-Vis spectroscopy. The oxygenation state of each sample was calculated using the following equation: Oxygenation state (%)=100*(Absₜ₀−Absₜ)/(Absₜ₀−Absₜₘᵢₙ) where Absₜ₀ and Absₜ represent the 430 nm absorbance at the starting point (t=0, complete deoxygenated state) and at the specific time, respectively, and Absₜₘᵢₙ represents the 430 nm absorbance at the minimum value.

Loading and Release Kinetics of Small Molecules

In order to load the negatively charged Mn-TPPS4, the chitosan surface of the RBC-polymer core was incubated with Mn-TPPS4 (2 mg/mL) for 4 hours under constant shaking. RBC membrane ghosts were then directly fused onto the Mn-TPPS4 loaded RBC-shaped polymer core. Note that for the extra polymer layer samples, the particles were resuspended in 1 mg/mL alginate solution under constant shaking for 0.5 hours and then the RBC membrane ghosts were fused onto the Mn-TPPS4 loaded RBC-shaped polymer core. For the positively charged doxorubicin (DOX) loading, the alginate surface of the RBC-polymer core was incubated for 4 hours in 3 mg/mL DOX under constant shaking. Then, the particles were resuspended in 1 mg/mL alginate solution under constant shaking for 5 minutes followed by the fusion of the RBC membrane ghosts onto the DOX loaded RBC-shaped polymer core. To quantify loading of Mn-TPPS4 and DOX, microplate reader UV-Vis measurements were obtained at 410 nm for Mn-TPPS4 and 475 nm for DOX. The Mn-TPPS4 loading capacity was found to be 3.5 μg/million particles while the DOX loading capacity was 5.5 μg/million particles. The dialysis bag diffusion method was used to evaluate Mn-TPPS4 and DOX release kinetics. Briefly, particles were loaded into 20 kDa MWCO Por Float-A-Lyzer G2 dialysis device, sealed in 50 mL conical tubes containing 20 mL phosphate-buffered saline (pH 7.4 or 5), and kept at 37° C. while stirring. At definite time points, 1 mL of dialysate was removed for absorbance analysis on a BioTek microplate reader and then 1 mL of the fresh dialysate solution was added to the conical tube. Each batch of experiments was performed in triplicate.

Magnetic Iron Oxide Nanoparticles Synthesis

Bare magnetic iron oxide (Fe₃O₄) nanoparticles were synthesized according to the previous method (Li et al., 2016). Briefly, 0.687 g of Fe(acac)₃ (1.94 mmol) was dissolved in 9 mL of benzyl alcohol. The solution was heated to 170° C. under reflux and stirring at 1500 rpm for 24 hours. After the reaction was cooled down to room temperature, 35 mL of EtOH was added into the mixture, and then centrifuged at 20000 rpm for 10 minutes. The supernatant was discarded, and the resulting precipitate was washed with EtOH twice to yield the required Fe₃O₄ nanoparticles. The synthesized Fe₃O₄ nanoparticles were stored in EtOH before use.

Loading of magnetic iron oxide nanoparticles into the RBC mimic particles

The bare Fe₃O₄ nanoparticles (NP) were incubated in 1 mg/mL chitosan solution overnight and then washed with DI water twice prior to the actual loading experiments. Chitosan coated Fe₃O₄ nanoparticles were then incubated with the alginate surface of the RBC-polymer core for several hours under constant shaking followed by another resuspension in alginate solution (1 mg/mL) under constant shaking for 0.5 hours to ensure optimal display of the alginate's negative charge. The RBC membrane ghosts were then fused onto the Fe₃O₄ loaded polyion to generate Fe₃O₄ loaded RBC mimics.

Confirmation of the Fe₃O₄ Nanoparticle Loading

The magnetic Fe₃O₄ NP loaded RBC mimic particles were suspended in an external magnetic field produced by a neodymium magnet. The bright field images were then obtained on the Leica DM13000 B inverted microscope to evaluate the magnetic guidance response.

Results and Discussion

Construction of RBC Mimic Particles

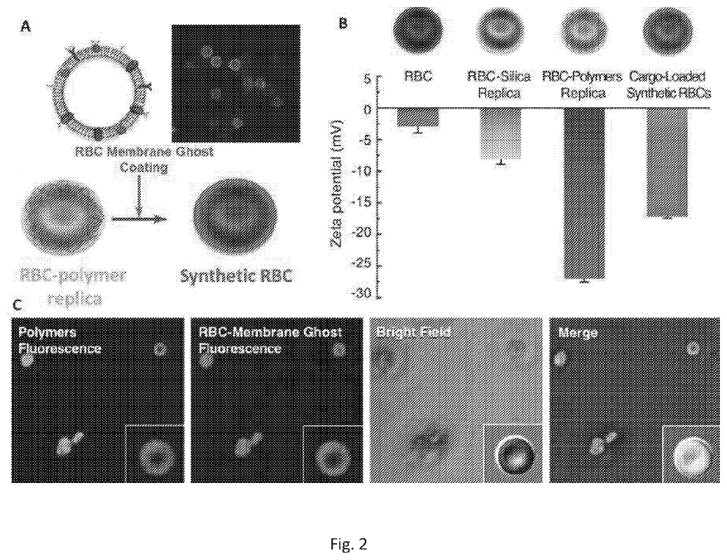

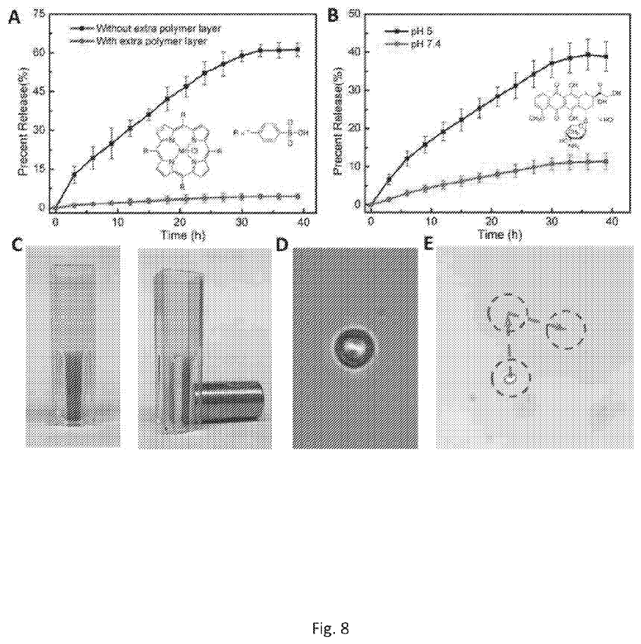

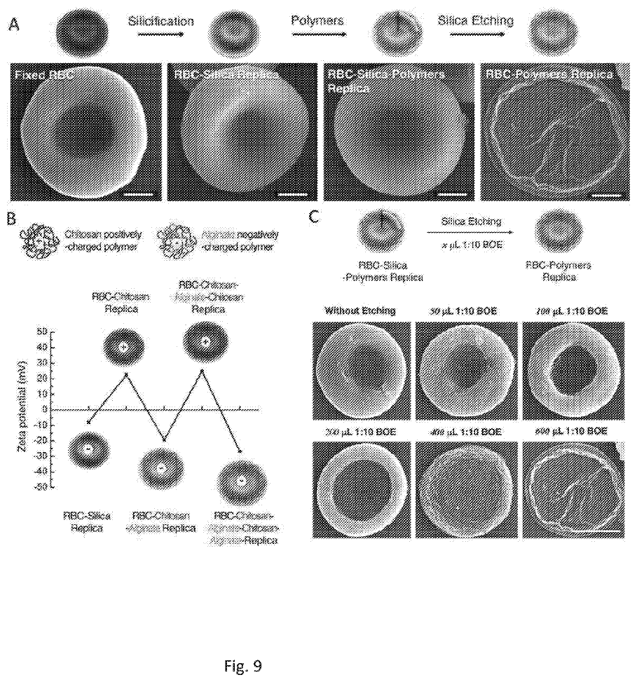

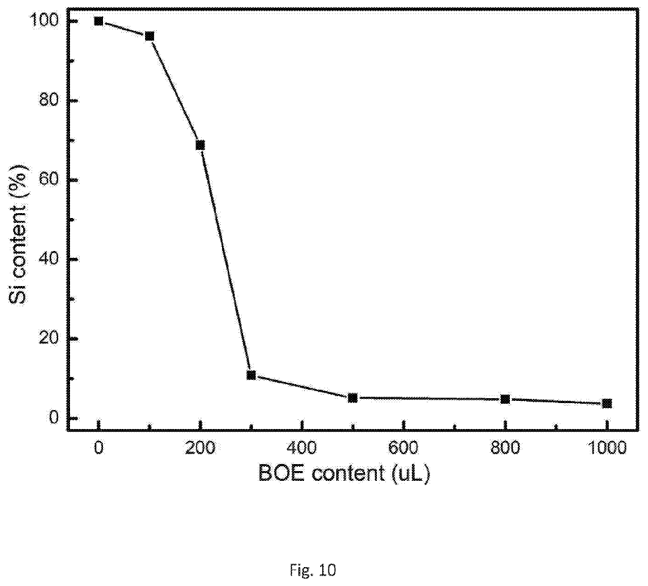

The design of functional artificial RBC or RBC mimic has three major steps; 1) preparing the silica RBC replica, 2) preparing the RBC-shaped polymer core, and 3) deriving RBC membrane ghosts from the natural RBCs and fusing it onto the surface of the RBC-shaped polymer core to yield a micron-size RBC mimic particle as shown in FIG. 1. Silica RBC replicas are prepared through the silica cell bio-replication process, which translates native RBCs into inorganic silica materials. In brief, purified RBCs are obtained and fixed with 4% formaldehyde solution in 1×PBS for at least 20 hours at room temperature and subsequently silicified at room temperature for 20 hours in a saline solution containing 100 mM silicic acid, adjusted to mildly acidic condition (pH 3.0). After a series of ethanol dehydration and drying, the silicified RBCs are calcined (500° C., 4 hours) to degrade the organics and yield silica RBC replicas (FIG. 8A) (Meyer et al., 2014). To construct the RBC-shaped polymer core, silica RBC replica is used as a template for the layer-by-layer self-assembly deposition of biocompatible complementary pclyelectrolytes (positively charged chitosan and negatively charged alginate polymers) onto the silica RBC replica surface through electrostatic interactions (FIG. 9B). Both chitosan and alginate are natural polysaccharides and are used in this process due to their low toxicity-allergenicity, high biocompatibility and biodegradability, that are important features for any potential biomedical applications (Lai & Lin, 2009; Lee & Mooney, 2012). The aminopolysaccharide chitosan layers are cross-linked using glutaraldehyde to provide stability to the mimic particles. The polymer coated silica RBC replica is then exposed to buffered hydrofluoric (HF) acid solution to etch the silica (FIG. 8C and FIG. 9) and yield RBC-shaped polymer core which due to its high surface charge, provides a platform for loading functional cargos through electrostatic interactions. The RBC-membrane ghosts are obtained by treating fresh RBCs with hypotonic solution to remove intracellular or cytosolic contents with minimal perturbations to the cell membrane (FIG. 2A) (Hu et al., 2011). The membrane ghosts are subsequently mixed with RBC-shaped polymer core and sonicated in cold water bath (about 0° C.) for 1 minute, during which the shear force imposed by the sonication process ruptures the RBC membrane ghosts and facilitate the fusion of the ghosts onto the surface of the RBC-shaped polymer core, giving rise to the final artificial RBC or RBC mimic particle, which is potentially loadable with small molecule cargoes, and a possible platform for biodetection and/or decontamination applications.

Characterization of RBC Mimic Particles

The RBC mimic particle was constructed as described above and characterized at different stages of the construction process. Zeta potential (ζ) measurements were obtained to assess fluctuations of the surface charge as functionalities were added to the native RBCs (ζ=−2.9 mV) to generate the RBC mimic particles. Upon silicification and subsequent calcination to yield silica RBC replicas, the zeta potential changed from −2.9 mV to −8.0 mV (FIG. 2B) consistent with the translation of the native RBC surface to the silanol groups (Si—OH) on the silica surface. When the polyion complex was added to the silica RBC replicas to generate RBC-shaped polymer core, further reduction in zeta potential from −8.0 mV to −27.0 mV was observed (FIG. 2B), consistent with the contributions of the alginate's negatively charged carboxylic acid groups (COOH). However, after fusing RBC membrane ghosts onto the polymer core surface, there was an increase in zeta potential from −27 mV to −17 mV (FIG. 2B), perhaps due to the charge screening by RBC membrane ghosts and negatively charged sialyl moieties on the RBC extracellular membrane side (Luk et al., 2014).

In order to verify the RBC mimic particle's structure, fluorescein isothiocyanate (FITC) modified chitosan was used to prepare RBC-shaped polymer core and then 1,2-dioleoyl-sn-glycero-3-phosphoethanolamine-N-(lissamine rhodamine B sulfonyl) (18:1 Liss Rhod PE) lipid was mixed with RBC-membrane ghosts prior to the fusion on the polyion template. The resulting dual-fluorophore-labeled RBC mimic particle was then visualized using confocal fluorescence microscopy. In FIG. 2C, microscopy images evidently show that different building blocks of RBC mimic particles overlap in the same location, revealing an intact core-membrane structure of the RBC mimic particles after the RBC membrane ghost fusion. In addition, the images also unequivocally revealed that RBC mimic particles designed as described, could maintain the characteristic biconcave discoid shape of the native RBCs, which provides excess surface area, and ultimately gives rise to the majority of the mechanical and transport properties of RBCs by enabling extreme shape deformations necessary for the cargo transport (Merkel et al., 2011).

Assessment of Flexibility and Deformation of RBC Mimic Particles

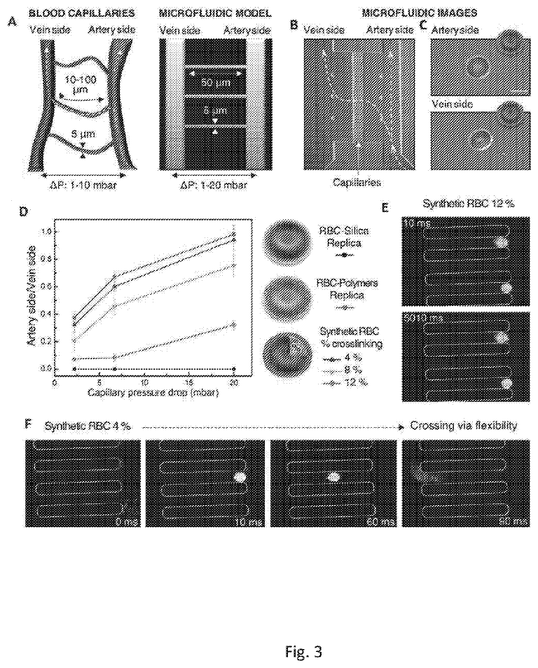

Having overcome the challenges inherent in designing the biconcave discoid shape of RBCs, other aspects of RBC-mimic material such as softness and deformability also needed to be considered. This is because flexibility-deformability of RBCs is one of the key properties that enable RBCs to easily traverse the microvasculature with dimensions smaller than their size and display long circulation times in vivo (Doshi et al., 2009; Merkel et al., 2011; She et al., 2013; Kozlovskaya et al., 2014). To demonstrate that the constructed RBC mimic particles are soft enough and can display deformability behavior akin to the native RBCs, a microfluidic blood capillary model (Kozlovskaya et al., 2014; Cui et al., 2014) was used to investigate the flow-based deformation of the RBC mimic particles (FIG. 3A). This model was designed using physiologically relevant dimensions and pressure drops that commonly occur in blood capillaries (Cui et al., 2014; Sun et al., 2015b).

In this design, RBC mimic particles were injected from a high-pressure inlet, while the control buffer (1×PBS) was injected from a low-pressure inlet. Since the flow rate of each inlet could be tuned independently, the pressure drops across the capillaries could easily be controlled (FIG. 3B). To tune the stiffness of RBC mimic particles and enable passage through the small capillary, different concentrations of glutaraldehyde cross-linker were used to crosslink the polymer layers of the RBC-shaped polymer core, based on the positive correlation between stiffness and changes in the amount of cross-linker used. It was observed that, the highly cross-linked RBC mimic particles could not pass through the capillaries, especially at lower pressure differentials (FIG. 3E), possibly due to higher stiffness and lower deformation capacity. On the contrary, RBC mimic particles with lower levels of cross-linking could undergo deformation and pass through the capillaries (FIG. 3F), confirming that softness and flexibility are important properties for the RBC mimic particles needed to traverse the smaller dimension microvasculature. Furthermore, the less cross-linked RBC mimic particles were also able to regain their discoidal shape upon exiting the capillary, further confirming the reversibility of the RBC mimic particle's shape deformation just like the native RBCs (FIG. 3C). The RBC mimic particle trajectories were further analyzed in the microfluidic blood capillary device to quantify the deformability behavior of the different particles (FIG. 3D). The ratio of the number of particles passing through the capillaries relative to the number of particles that stayed on the sample side (Cui et al., 2014; Sun et al., 2015b) was calculated. This was based on the principle that, particles that could easily pass through the capillaries would have a higher ratio, while the particles that could not pass through would have a ratio of zero (Cui et al., 2014; Sun et al., 2015b). The decrease in cross-linker concentration resulted in decreased stiffness of the RBC mimic particles, and corresponding increase in softness and deformation capacity. As expected, even at higher pressure drops, the control silica RBC replica could not pass through the capillaries, possibly due to the stiffness of silica network. Further, RBC mimic particles with 4% cross-linker concentration behaved similar to the native RBCs in the microfluidic blood capillary model, suggesting that RBC mimic particles at this cross-linker concentration may show similar in vivo circulation properties as native RBCs.

Assessment of the Membrane Properties of RBC Mimic Particles

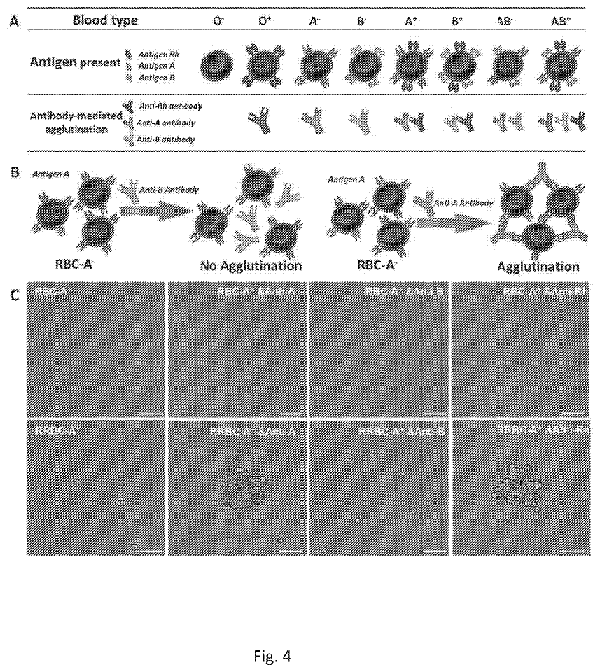

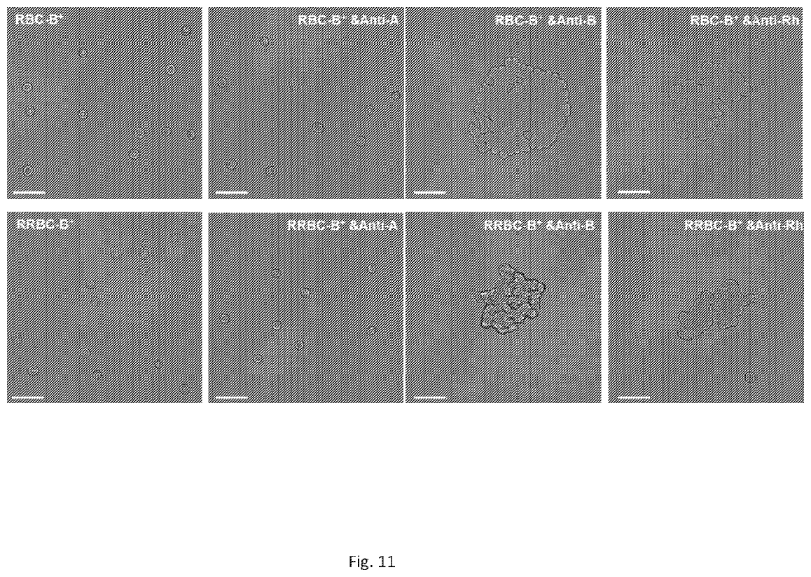

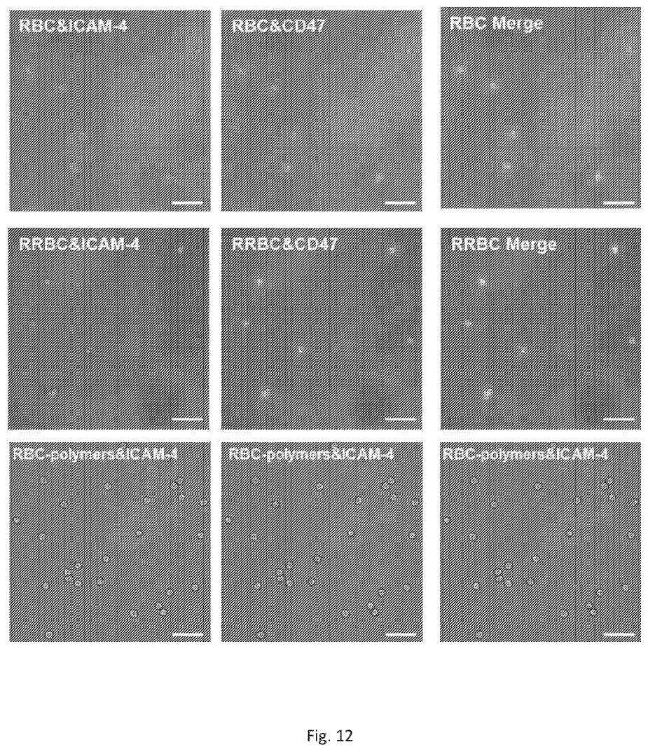

RBC membrane is highly oriented with asymmetric distribution of phospholipids, glycans and proteins. The glycans or proteins on the extracellular side, play many roles in phagocytic cell recognition and internalization (Luk et al., 2014; Cohen et al., 2009). Similarly, right-side-out-membrane orientation of the RBC mimic is important in maintaining the same surface property as the native RBC (Luk et al., 2014). In order to verify the membrane orientation of the RBC mimic, antibody-mediated agglutination assay was performed to detect the classical AB(O) and Rh antigens (FIG. 4A). These antigens are the glycosphingolipid protruding from the RBC membrane and the lipoprotein embedded in the RBC membrane respectively. Commercially prepared anti-A, anti-B, and anti-RhD sera were used to examine these antigens (Park et al., 2017; Wang et al., 2014) on both the native and RBC mimic membranes When treated with their respective anti-type sera, both native RBC and the RBC mimic rapidly agglutinated unlike when they were treated with different anti-type sera (FIG. 4C and FIG. 11). This was attributed to the preservation of the native RBC membrane biomarkers on the RBC mimics together with the right-side-out membrane orientation. In addition, ICAM-4, an erythroid-specific membrane glycoprotein which plays an important role in the RBC's interactions with macrophage cells (de Back et al., 2014), was also detected by the fluorescent anti-ICAM-4 antibody probe (FIG. 12), further confirming RBC mimic's membrane protein preservation and the right-side-out membrane orientation.

In addition to the self-antigens, immunosuppressive proteins are another important group of RBC membrane biomarkers that protect the RBCs from clearance/destruction by phagocytic cells (Su et al., 2016; Hu et al., 2011). Specifically, CD47 is a well-documented protein marker firmly embedded within the RBC membrane and inhibits macrophage phagocytosis of RBCs through interactions with the signal regulatory protein alpha (SIRPa) receptor (Hu et al., 2011). We used FITC conjugated anti-CD47 antibody to confirm the presence of the CD47 protein on the rebuilt RBC-like particle membrane and observed that, the CD47 protein could still be recognized by the antibody probe (FIG. 12), indicating that the immunosuppressive proteins were not perturbed during the construction of the RBC mimic further supporting the right-side-out-membrane orientation. The presence of these immunosuppressive proteins is expected to lead to long in vivo circulation times and possible better delivery of the intended cargoes by the RBC mimic particles (Su et al., 2016; Hu et al., 2011).

Elucidation of the Circulation Properties of RBC Mimic Particles

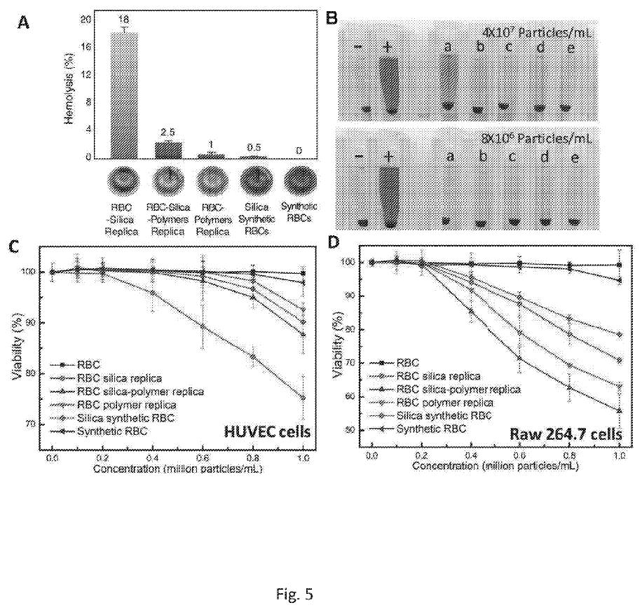

The circulation or vascular flow of the RBC mimic particles was tested using the Ex ovo chick chorioallantoic membrane (CAM) model (Townsen et al., 2013; Surfee et al., 2016). Before testing the circulation properties, the biocompatibilities of the various RBC mimic particles were assessed by carrying out hemolysis and in vitro cytotoxicity assays using human umbilical vein endothelium cells (HUVEC) and mouse macrophage Raw264.7 cells. Hemolysis test is an important indicator of in vitro blood compatibility of a biomaterial and is often carded out because many biomedical applications require intravenous injection of the biomaterials (Townsen et al., 2013; Durfee et al., 2016). In our hemolysis analyses using about 8×10⁶ particles/mL of the test materials (silica RBC replicas, silica RBC replica @Polymer, RBC-shaped polymer core, silica RBC replica@polymer-RBC ghost, and RBC mimic particle) against 4×10⁸/mL native RBCs, only silica RBC replica exhibited significant hemolytic activity relative to the etched silica or polymer coated particles (FIG. 5A). However, when a higher concentration of the test materials (about 4×10⁷ particles/mL) was used, only RBC mimic particles did not cause hemolysis, confirming that bare silica and to a lesser extent alginate surfaces are toxic to red blood cells in vitro, while fusion with RBC ghosts abolishes this toxicity to RBCs (FIG. 5A). This is a significant observation because upon intravenous injection, biomaterials establish direct contact with the blood vessels making it necessary to understand the biocompatibility of biomaterial to relevant cells in the blood vessels, especially the endothelial cells that cover the blood vessel lumen (Fang et al., 2013). We carried out further in vitro cytotoxicity tests of the RBC mimic particles against HUVEC and Raw264.7 cells. Using CellTiter-Glo® 2.0 cell viability assay kit to ascertain the viability of these cells incubated with the RBC mimic particles for 24 hours, we observed no loss of cell viability congruent with the hemolysis findings (FIG. 5B, 5C), further corroborating the high biocompatiblility of the RBC mimics.

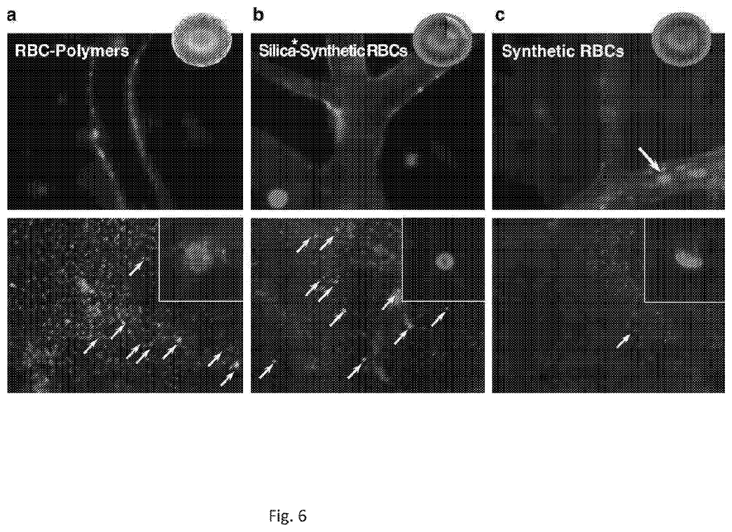

Having established that RBC mimic particles are biocompatible and have no hemolytic effects, the vascular flow of these particles was tested by injecting them into the vasculature of the Ex ovo chick embryo (CAM) and used direct intravital imaging to assess the vascular flow or circulation of the RBC mimic particles. Significantly, while the RBC mimic particles were able to easily circulate within the deep blood vessels of the Ex ovo chick embryo, both the RBC-shaped polymer cores and silica RBC replica@polymer-RBC ghost were rapidly arrested in the CAM capillary bed (FIG. 6), possibly, due to the combined roles of the native red blood cell membrane properties and the stiffness caused by the silica network. The circulation of the RBC mimic particles was only possible when these particles were disguised as the native RBCs through a process of successive silica etching followed by fusion of native RBC membrane ghosts to generate a complex with sufficiently low modulus and native RBC membrane like properties capable of withstanding long-term circulation times.

Demonstration of the Oxygen Carrying Capability of RBC Mimic Particles

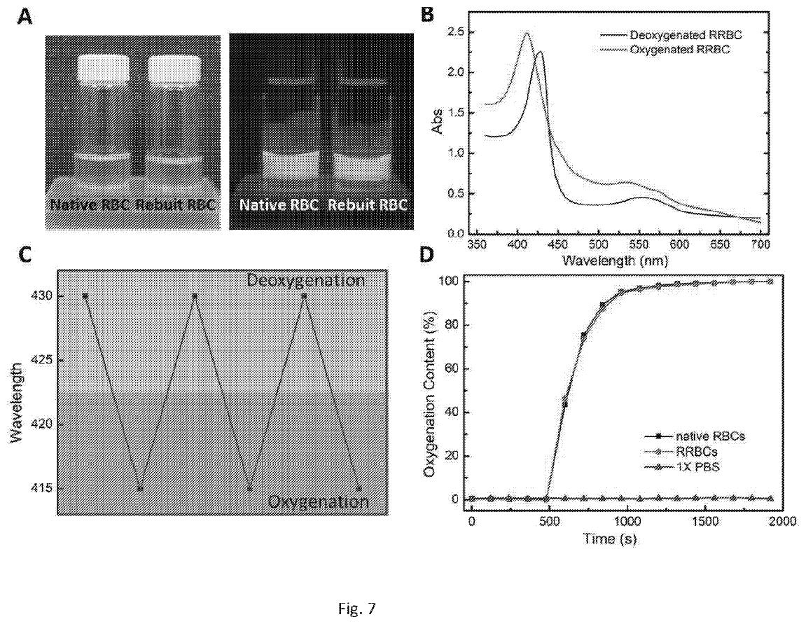

One of the most important functions of the RBCs is to transport oxygen from the lungs to the body tissues with the help of the heme containing hemoglobin (Hb) which reversibly binds oxygen in the blood to form oxygenated Hb (HbO2) (Modery-Pawlowski et al., 2013; Jia et al., 2012; Duan et al., 2012). In order to fully mimic the native RBC, hemoglobin was loaded on to the RBC mimics to yield oxygen-carrying material. Before directly investigating whether this hemoglobin loaded RBC could carry oxygen, luminol based chemiluminescence was used to reveal the presence of hemoglobin in the RBC mimic particles (Doshi et al., 2009). Luminol is commonly used in forensics as a diagnostic tool for the detection of blood stains (Bochev et al., 1993). In theory, when hemoglobin and the luminol-perborate mixture come into contact, the iron in the hemoglobin accelerates the reaction of luminal with the peroxide generated from perborate to produce a bluish glowing compound, indicating that hemoglobin is present (Doshi et al., 2009; Bochev et al., 1993). FIG. 7A shows that both the native RBCs and RBC mimic can generate bluish glow when tested with luminol in the dark, demonstrating that hemoglobin inside the RBC mimics can still show the iron catalytic property. To investigate the oxygen carrying capability of the RBC mimic, UV-Vis absorption analysis was used to reveal the reversible shift of maximum absorption peaks of RBCs in oxygenated and deoxygenated states (Modery-Pawlowski et al., 2013; Jia et al., 2012; Duan et al., 2012). The characteristic absorption peak of the native RBC and RBC mimic in oxygenated states appeared at 415 nm (FIG. 7B) which after bubbling nitrogen for 2 hours and adding reducing agent sodium dithionite (Na₂S₂O₄), red-shifted to 430 nm (FIG. 7B), confirming the deoxygenated states of the native RBCs and the RBC mimic. These deoxygenated RBC mimics could carry oxygen after exposure to atmospheric oxygen. This process of binding and releasing oxygen could be repeated several times (FIG. 7C), indicating that our RBC mimic has a similar oxygen carrying capability as the native RBCs. To further investigate the oxygen carrier capability of the RBC mimic, the oxygenation rate of the deoxygenated sample was monitored by observing the absorbance change at 430 nm. The oxygenation curve for the native RBCs and the RBC mimics had a similar oxygen curve profile (FIG. 7D), further confirming that, hemoglobin loaded RBC mimic can be a possible substitute for the native RBCs.

Multifunctional Cargo Loading of RBC Mimic Particles

Due to the similar property of the RBC mimics as the native RBCs, by loading various functional cargoes, multifunctional RBC mimics can be efficiently generated to advance their applications in medicament delivery and bio-detection analyses. The first example of this multifunctional creation is the ability to package RBC mimic with small biofunctional molecules. A potential magnetic resonance imaging (MRI) contrast agent 5,10,15,20-Tetrakis(4-sulfonatophenyl)-21H,23H-porphine manganese(III) chloride (Mn-TPPS4) and anticancer chemotherapy drug, doxorubicin hydrochloride (DOX) were loaded into the functional layer of the RBC mimic and then investigated for the release profiles. The drug release was examined by dialyzing the samples in 1×PBS for 40 hours at 37° C., recording the absorbance (412 nm for Mn-TPPS4 and 485 nm for DOX) of the supernatant collected at different time points to determine the quantity of small molecules released under these conditions. After directly fusing the RBC membrane ghosts, the total Mn-TPPS4 released was about 60% after 40 hours (FIG. 8A). However, in the presence of an extra polymer layer between the loaded Mn-TPPS4 and fused RBC membrane, only about 5% of Mn-TPPS4 could be released from the RBC mimic (FIG. 8A). This distinct and significant decrease was attributed to an increase in complexity of diffusion pathway by the extra polymer layer. The negligible release of Mn-TPPS4 could help the RBC mimic to have potential applications in medical imaging (Liang et al., 2014). Analysis of the DOX release behaviors revealed greater total DOX release at pH 5.0 (about 40%) compared to pH 7.4 (about 10%) after 40 hours (FIG. 8B), possibly due to the carbon/late groups on the alginate layer becoming less negatively charged at lower pH, which theoretically, should reduce the electrostatic interactions between the alginate layer and DOX, leading to increased DOX release kinetics/profiles (Gao et al., 2017). This pH sensitivity suggested a potential lower toxicity of DOX to normal tissues because of limited drug release under physiological conditions (around pH 7.0) versus potentially better anticancer activity of the DOX loaded RBC mimic in the acidic (around pH 5.0) tumor environment due to higher DOX release. Besides small molecules, nanosized particles could also be loaded onto the RBC mimics. To prove this, magnetically active RBC mimic was fabricated by packaging it with magnetic iron oxide (Fe₃O₄) nanoparticles. By controlling the external magnetic field, the magnetic RBC mimic exhibited “on” and “off” magnetic guidance/movements (FIG. 8D, 8E), indicating that these magnetically active RBC mimics may be used for magnetic resonance imaging or as micromotors.

### Conclusion

In summary, findings are presented on the construction of RBC mimic particles based on mimicking the unique characteristics of the native RBCs. The RBC mimic particles were constructed through three separate building blocks involving silica cell bio-replication process, layer-by-layer deposition of biocompatible polymers to translate native RBCs into flexible RBC-shaped polymer core which is loadable with functional cargos to provide special functions and encapsulation of the RBC derived membrane ghosts that mimic the surface properties of the native RBCs. The RBC mimic particle displays deformable property, zero hemolytic activity, low cytotoxicity and vascular flow in the Ex Ovo chick embryo vasculature. In addition, different functional cargos (e.g., hemoglobin, Mn-TPPS4, DOX, iron oxide nanoparticles) can be loaded onto the RBC mimic particle to enable oxygen delivery capability, MRI contrast imaging, and therapeutic drug delivery. A robust RBC mimic particle was produced that may serve as a great tool to promote the understanding of complex life processes and be used as a model for designing multifunctional delivery and bio-detection platforms.

### Example 2

Red blood cells (RBCs) possess unique characteristics relative to other cells, making them attractive targets for cellular biomimicry. Here a micron-sized multifunctional RBC replica (rebuilt RBC) is designed and constructed using the successive steps of silica bio-replication, calcination, polyion polymer layer-by-layer deposition, desilicification, and fusion of RBC membrane ‘ghosts’. The rebuilt RBC displays biconcave shape, deformability, zero hemolytic activity, and vascular flow in ex ovo chick embryo and in vivo mouse models. The rebuilt RBC is also loadable with various cargos including hemoglobin for oxygen transport, contrast agents for magnetic target localization or magnetic resonance imaging applications, therapeutic small molecules, and a biosensor for pore-forming toxin detection. Overall, rebuilt RBCs represent a new, robust, long circulating synthetic platform for use in therapy bio-detection, and imaging as well as a unique tool to advance understanding of complex life processes.

Augmenting the intrinsic functionality of cells remains a poorly understood subject due to the inherent complexity and fragility of biological systems (Sun et al., 2015). To overcome these challenges, artificial cells are designed to simplify and mimic functions of natural cells as well as provide a platform to incorporate abiotic features not innate to the native cells (Xu et al., 2016). Given their low biologic complexity (e.g., lack of cell nucleus and most intracellular organelles (Shane et al., 2014)), red blood cells serve as a suitable target for biomimicry and rebuilding of functional artificial cells. While biologically simple, RBCs possess unique potential focal points for biomimicry such as their special biconcave discoidal shape that provides a favorable surface area-to-volume ratio and allows RBCs to undergo remarkable flexible deformations, their ability to carry oxygen through the formation of the oxyhemoglobin complex, and the presence of multiple molecular biomarkers on the membrane including CD47 that ensure recognition as self by the immune surveillance system, leading to long circulation times (Merkel et la., 2011).

There have been several attempts to generate artificial RBC-like materials by mimicking the key structural and functional features of RBCs. Mitragotria, et al. (Doshi et al., 2009) developed synthetic biomaterial particles that closely mimic the shape, flexibility, and the ability to carry oxygen similar to the natural RBCs. Similarly, DeSimone, et al. (Merkel et al., 2011) explored the effect of mimicking RBC shape and flexibility on the vascular circulation time and biodistribution characteristics. While these studies presented encouraging findings, their overriding shortcoming was a predominant focus on one or two aspects of the RBC's unique characteristics, rather than a broad spectrum of RBC properties that can lead to a multifunctional RBC-mimicking material. In addition, neither of these studies considered the unique properties of the RBC membrane and how they impact long-term circulation times nor did they attempt to provide RBCs with augmented non-native functions. In this study, a modular design approach (She et al., 2013; Kozlovskaya et al., 2014) was used to construct a multifunctional artificial rebuilt RBC that fully mimics most of the broad properties of native RBCs and performs additional abiotic functions not inherent to the native RBCs. This modular design strategy is based on the use of three nanoscale synthesis procedures: silica cell bio-replication, layer-by-layer self-assembly, and encapsulation within an RBC derived membrane ghost (FIG. 12). It is envisioned that this rebuilt RBC (RRBC) platform will provide a better understanding of the relationship between structure and functions of RBCs, and further advance our understanding of complex living systems.

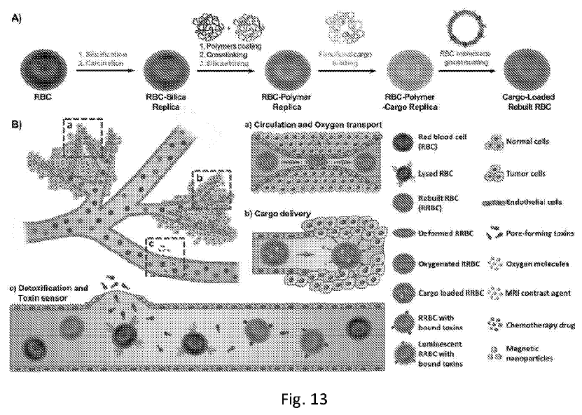

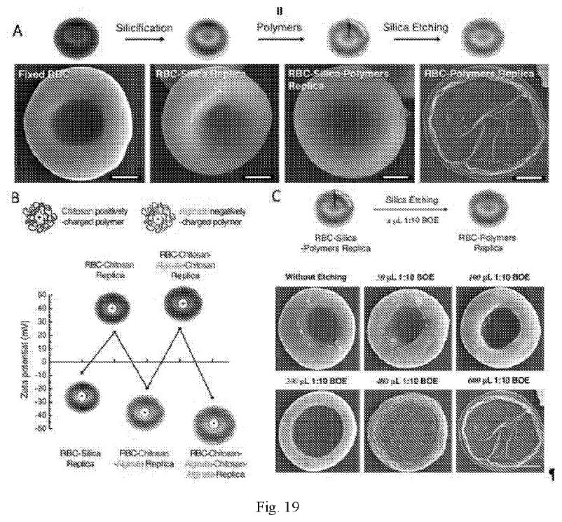

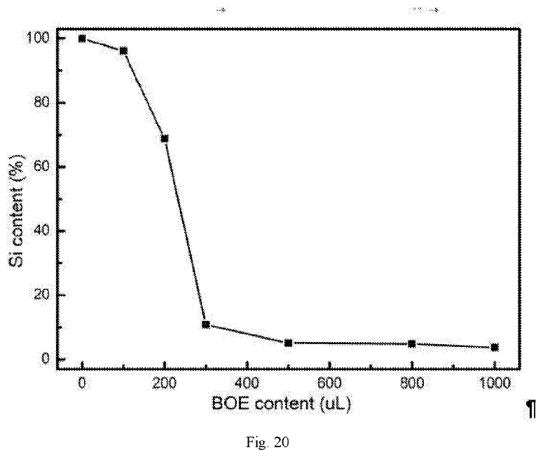

In order to construct the RRBC, four successive steps were used (FIG. 13); 1) preparation of a silica RBC replica, 2) polymer coating and silica removal to create a RBC-polymer replica, 3) functional cargo loading, and 4) fusing of a purified RBC membrane ghost onto the surface of the RBC-polymer replica to yield a micron-size RRBC particle with nearly identical size and shape as the native RBC (FIG. 19A). As detailed in the methods section, the silica RBC replica was prepared through the silica cell bio-replication process, which under mild acidic conditions (Meyer et al., 2014; Kaehr et al., 2012). translates native RBCs into inorganic silica replicas composed of about 10-nm amophous silica films deposited over all biomolecular interfaces both within and on the cell surface. To construct the RBC-shaped polymer core, the silica RBC replica was used as a template for layer-by-layer self-assembly of biocompatible complementary polyelectrolytes (positively charged chitosan and negatively charged alginate polymers) onto the silica RBC replica surface through attractive electrostatic interactions (FIG. 19). Both chitosan and alginate are natural polysaccharides and were used in this process due to their combined biomedically relevant features of low toxicity-allergenicity, high biocompatibility and, biodegradability (Lai & Lin, 2009; Lee & Mooney, 2012). The aminopolysaccharide chitosan layers were cross-linked to differing extents using glutaraldehyde to provide stability to the RRBC particles. The polymer-coated silica RBC replica (FIG. 14A) was then suspended in a buffered hydrofluoric (HF) acid solution to slowly and controllably etch the silica core (FIGS. 19 and 20) yielding a RBC-polymer replica. As shown in FIG. 19C, the silica replica etches from the inside out leaving a silica/polymer rim that preserves the RBC shape. Due to their high surface charge, the polymer layers provide a reservoir for loading functional cargos through electrostatic interactions. The RBC-membrane ghost was obtained by treating fresh RBCs with a hypotonic solution to remove intracellular or cytosolic contents with minimal perturbations to the cell membrane (FIG. 14B) (Hu et al., 2011; Luk et al., 2014). The membrane ghost was then subsequently mixed with RBC-polymer replica and sonicated in an ice water bath (about 0° C.) for 1 minute, so that the shear force imposed by the sonication process could rupture the RBC membrane ghosts and facilitate their fusion onto the surface of the RBC-polymer replica, yielding the final RRBC.

Different strategies were used to characterize the RRBC during the construction process. Zeta potential (ζ) measurements were obtained to assess the progressive variation of surface charge after each synthetic step beginning with the native RBCs (ζ=−2.9 mV) (FIG. 19B). Upon RBC silicification and subsequent calcination to yield silica RBC replicas, the zeta potential values changed from −2.9 to −8.0 mV (FIG. 14E) consistent with the translation of the native RBC surface to amophous silica where the acidity of surface silanol groups (Si—OH) increases the magnitude of the negative charge. Layer-by-layer deposition of cationic chitosan followed by anionic alginate resulted in alternating positive and negative zeta-potentials with increasing magnitudes (Figure S1B). After four ALD layers the zeta potential was reduced from −8.0 to −27.0 mV was observed (FIG. 14E), consistent with the contributions of the negatively charged carboxylic acid groups (COOH) present in the final alginate layer. However, after fusing RBC membrane ghosts onto the polymer core surface, there was an increase in zeta potential from −27 to −17 mV (FIG. 14E), attributed to the charge screening effects of the RBC membrane ghosts and the negatively charged sialyl moieties on the extracellular side of the RBC membrane (Hu et al., 2011; Luk et al., 2014). Furthermore, when transferred to the biologically relevant solution, 154 mM NaCl, utilized to assess the native RBC zeta potential, the RRBC zeta potential (−3.4 mV) closely matched that of the native RBC (−2.9 my) (FIG. 14E).

In order to verify the RRBC particle's structure, fluorescein isothiocyanate (FITC) modified chitosan was used to prepare the RBC-shaped polymer replica and then fluorescent 1,2-dioleoyl-sn-glycero-3-phosphoethanolamine-N-(lissamine rhodamine B sulfonyl) (18:1 Liss Rhod PE) lipid was mixed with the RBC-membrane ghosts and then fused onto the RBC-shaped polymer replica. The resulting dual-labeled RRBC particle was then visualized using confocal fluorescence microscopy. In FIG. 14, microscopy images show that different building blocks of RRBC particles overlap in the same location, revealing an intact core-membrane structure of the RRBC particles with the encapsulating RBC membrane ghost. In addition, the images also unequivocally reveal that RRBC particles could maintain the characteristic biconcave discoid shape typical of the native RBCs, which provides excess surface area and ultimately gives rise to the majority of the mechanical and transport properties of the RBCs by enabling necessary extreme shape deformation (Meyer et al., 2014; Kaehr et al., 2012).

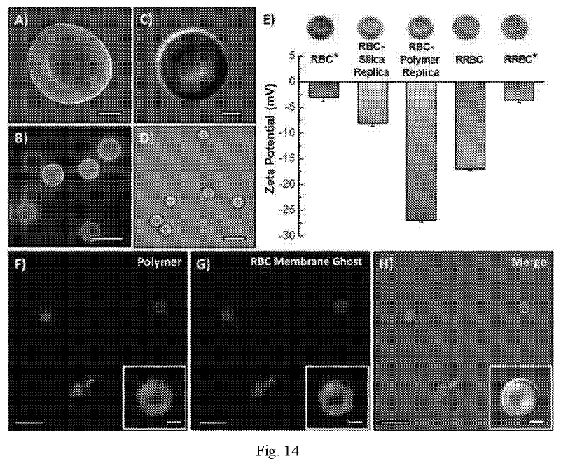

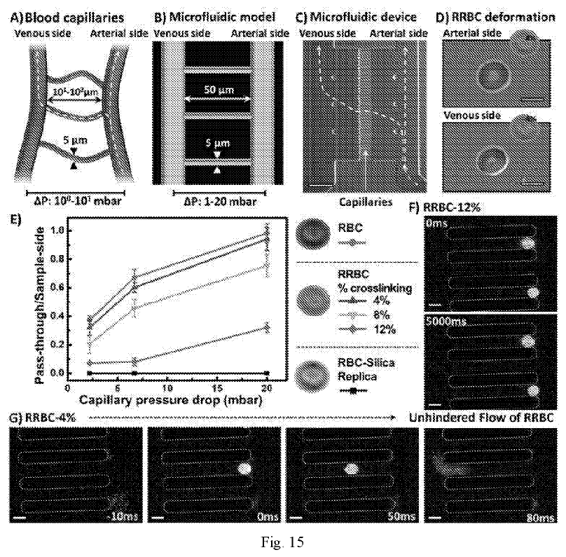

Having overcome the challenges inherent in designing the biconcave discoid shape of the RBCs, the softness and deformability behavior of the RRBC particle was then examined. Flexibility-deformability of the native RBCs is one of the key properties that enables RBCs to easily traverse the microvasculature with dimensions smaller than their size and display long circulation times in the body (Doshi et al., 2009; Merkel et al., 2011; Su et al., 2016; She et al., 2013; Kozlovskaya et al., 2014). A microfluidic blood capillary model (Cui et al., 2014; Sun et al., 2015) designed using physiologically relevant dimensions and pressure drops that commonly occur in blood capillaries (Cui et al., 2014; Sun et al., 2015), was used to investigate the flow-based deformation of the RRBC particles (FIGS. 15A and 15B). RRBC particles were injected from a arterial inlet, while the control buffer (1×PBS) was injected from a venous inlet (FIG. 15C) of the capillary model. Since the flow rate of each inlet could be tuned independently, the pressure drops across the capillaries could easily be controlled. To tune the stiffness of the RRBC particles and enable their passage through the small capillary, different concentrations of the glutaraldehyde crosslinker were used to link the polymer layers of the RBC-shaped polymer core, based on the positive correlation between particle stiffness and changes in the amount of crosslinker used (Doshi et al., 2009; Merkel et al., 2011; Su et al., 2016). It was observed that, the highly cross-linked RRBC particles could not pass through the model capillaries at lower pressure differentials (FIG. 15F), due to higher stiffness and lower deformation capacity. On the contrary, RRBC particles with lower cross-linking levels could undergo the requisite deformation and pass through the model capillaries (FIG. 15G), confirming that softness and flexibility are important properties needed for the RRBC particles to traverse smaller dimension microvasculature. Furthermore, the less cross-linked RRBC particles were able to regain their discoidal shape upon exiting the capillary flow system (FIG. 15D), confirming the reversibility of the RRBC particle shape deformation characteristic of the native RBCs. The trajectories of the particles in the microfluidic blood capillary device were further analyzed to quantify the deformability behavior (FIG. 15E). This was done by calculating the ratio of the number of particles passing through the capillaries relative to the number of particles remaining on the sample side (Cui et al., 2014; Sun et al., 2015), based on the principle that, the particles that could easily pass through the capillaries would have a higher ratio, while the particles that could not pass through would have a zero ratio (Cui et al., 2014; Sun et al., 2015). A decrease in glutraldehyde crosslinker concentration resulted in decreased stiffness of the RRBC particles, and a corresponding increase in softness and deformation capacity (FIG. 15E). As expected, even at higher pressure drops, the control silica RBC replica could not pass through the capillaries, possibly due to the stiffness of silica network. Further, RRBC particles with 4% crosslinker concentration and the native RBCs had similar deformability behavior in the microfluidic blood capillary model, confirming that RRBC particles at this crosslinker concentration may display similar in vitro circulation properties as the native RBCs.

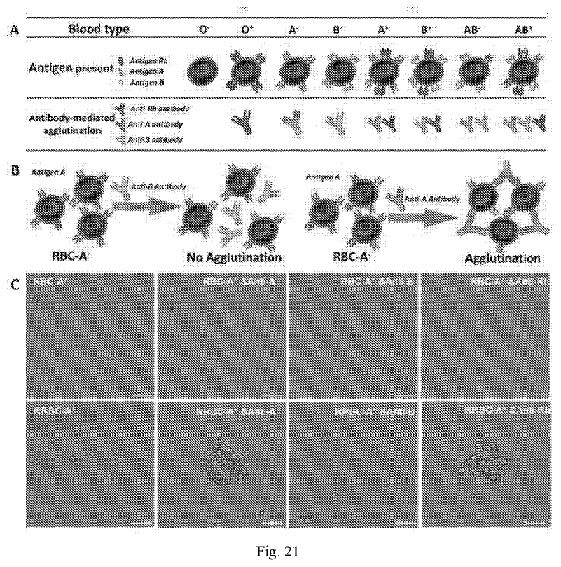

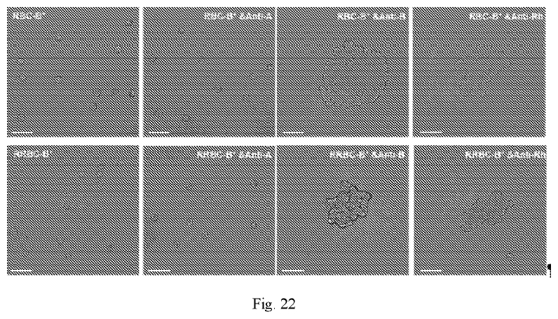

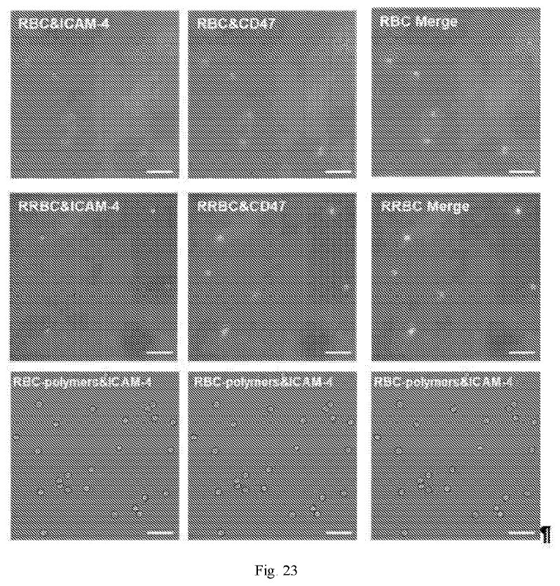

Having confirmed the deformability characteristics of the RRBC particles in the capillary model, their membrane properties were then assessed. The membrane of the native RBC is highly oriented with asymmetric distribution of phospholipids, glycans, and proteins between the intracellular and extracellular sides (Cohen et al., 2009; Park et al., 2017; Wang et al., 2014). The glycans and proteins on the extracellular side, play important roles in phagocytic cell recognition and internalization (Luk et al., 2014; Cohen et al., 2009; Park et al., 2017; Wang et al., 2014). Similarly, right-side-out-membrane orientation of the RRBC particle is critical for this construct to maintain native RBC membrane-like properties (Luk et al., 2014). In order to verify the membrane orientation of the RRBC particle, an antibody-mediated agglutination assay was performed to detect the classical AB(O) and Rh antigens (FIG. 21). While AB(O) are glycosphingolipid antigens protruding from the RBC membrane, Rh is a lipoprotein antigen embedded in the RBC membrane (Cohen et al., 2009; Park et al., 2017; Wang et al., 2014). Commercially sourced anti-A, anti-B, and anti-RhD sera were used to examine the antigens (Cohen et al., 2009; Park et al., 2017; Wang et al., 2014) on the membranes of both the native RBC and RRBC particle. When treated with their respective anti-type sera, both the native RBC and the RRBC particle rapidly agglutinated (FIG. 21), whereas no agglutination occurred when they were treated with non-matching anti-type sera (FIGS. 21 and 22). This was attributed to the preservation of the RBC membrane biomarkers together with the right-side-out membrane orientation. In addition, the presence and surface membrane availability of the ICAM-4, an erythroid-specific membrane glycoprotein important in the RBC's interactions with macrophage cells (de Back et al., 2014), was also confirmed by the fluorescent anti-ICAM-4 antibody (FIG. 23), which further speaks to the RRBC particle's membrane protein preservation and the right-side-out membrane orientation. In addition to the self-antigens, the presence of immunosuppressive proteins (e.g. CD47) was also assessed (Hu et al., 2011; Luk et al., 2014). CD47 is a transmembrane protein whose presence on the surface of the RBC membrane inhibits macrophage phagocytosis of RBCs and ensures long circulation times (Hu et al., 2011; Luk et al., 2014). Using a FITC conjugated anti-CD47 antibody, the presence of CD47 protein on the membrane of the RRBC particles was confirmed (FIG. 23), which together with the self-antigens and ICAM-4, indicates that the status of the immunosuppressive proteins was not perturbed/destroyed by the construction of the RRBCs. Thus, the presence of these immunosuppressive proteins on the RRBC membrane should lead to long in vivo circulation times within the vascular network (Hu et al., 2011; Luk et al., 2014) and possibly ensure better delivery of the intended cargos.

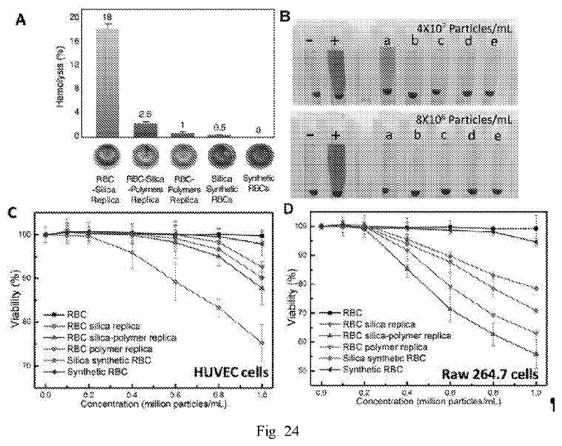

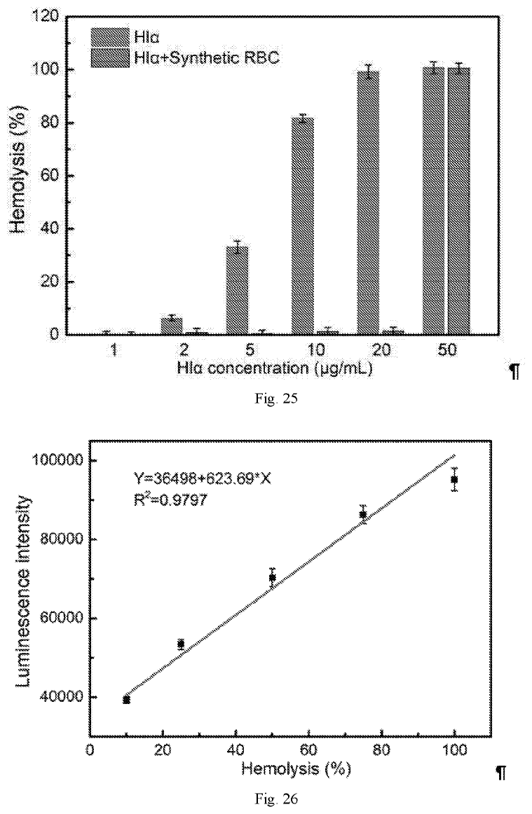

To investigate the ability of RRBCs to flow within a vascular network in a living system, the ex ovo chick chorioallantoic membrane (CAM) model and in vivo mouse models were used in conjuction with appropriate imaging techniques. However, before testing the vascular flow behavior of the RRBCs, the biocompatibility characteristics of the RRBCs along with the RBC silica replicas, RBC silica/polymer replicas, RBC-polymer replicas, and silica-RBC replicas were first assessed using a hemolysis assay (Townson et al., 2013; Durfee et al., 2016) (FIG. 24). In the hemolysis assays, it was determined that only RBC silica replicas exhibited significant hemolytic activity compared to the other samples at biologically relevant concentrations (FIG. 24). However, when a very high concentration of the test materials (about 4×10⁷ particles/mL) was used, only the complete RRBC did not cause hemolysis while the rest of the test materials exhibited varying extents of hemolysis (FIG. 24), confirming that bare silica and to a lesser extent alginate surfaces are somewhat toxic to red blood cells in vitro, while fusion with RBC ghosts abolishes this toxicity. Following confirmation of biocompatibility with native RBCs, cytotoxicity to other cells likely to be encountered by RRBC was assessed. Human umbilical vein endothelial cells (HUVEC) and mouse macrophage Raw264.7 cells were utilized for these assays. Cytotoxicity tests revealed no loss of cell viability in both HUVEC and Raw264.7 cells after exposure to RRBC particles, congruent with the hemolysis test results (FIG. 24) and corroborating the overall high biocompatibility of the RRBC particles. These findings are significant because upon intravenous injection, biomaterials establish direct contact with the blood vessels which makes it necessary to understand their biocompatibility with the endothelial cells that cover the blood vessel lumen (Fang et al., 2013).

Having established that the RRBCs exhibit excellent biocompatibility, their vascular flow characteristics within living systems were then assessed. Using the ex ovo chick embryo (CAM) and direct intravital imaging (Townson et al., 2013; Durfee et al., 2016), it was observed that, while the RRBC particles were able to easily circulate within the deep blood vessels of the ex ovo chick embryo, both the RBC-polymer replica without native RBC membrane coating and silica*-RRBC (silica RBC replica©polymer-RBC ghost, without etching) were rapidly arrested in the CAM capillary bed (FIGS. 16G and 16H), due to the absence of self recognition proteins or increased stiffness due to the silica core, respectively. Importantly, it should be noted that, the circulation of the RRBC particles was only possible when they were closely matched in physical properties to the native RBCs through a process of successive silica etching followed by fusion of the native RBC membrane ghosts (FIG. 16) to generate a complex with sufficiently low modulus and native RBC membrane like properties capable of withstanding inherent challenges of long-term circulation.

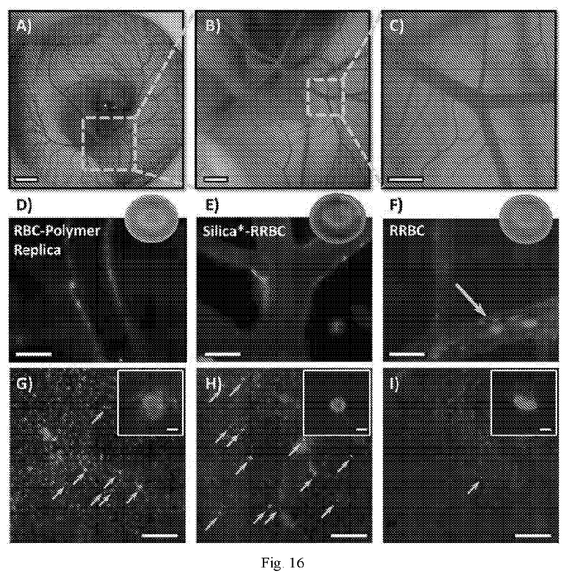

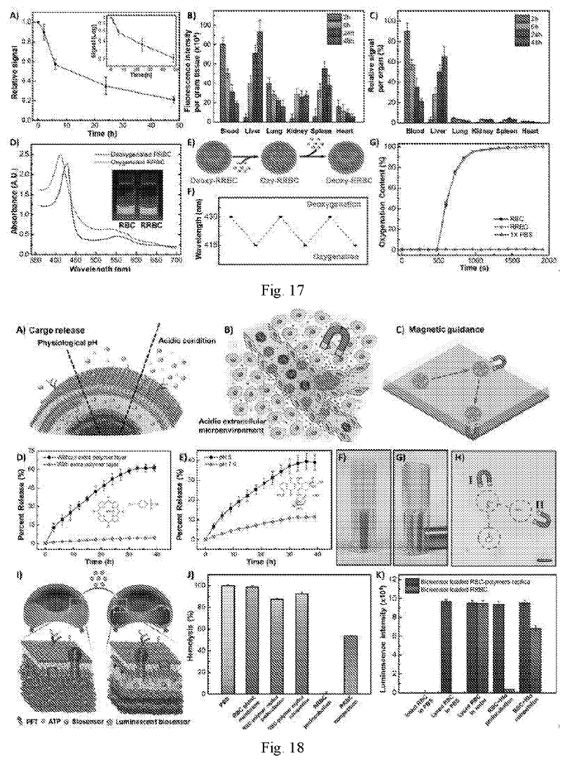

Further characterization of the vascular flow properties of RRBC particles was carried out by examining the pharmacokinetic behavior and biodistribution profiles of the RRBC particles in in vivo in a mouse model. In order to avoid potential immune responses caused by the blood type non-conformity, the RRBC particles used for this characterization were prepared using syngeneic mice RBCs. FITC-labeled RRBC particles (3×10⁷ particles/mL, 100 μL) were administered by retro-orbital injection. Blood was collected at 24 and 48 hours post-injection, the RBC-membrane-coated particles exhibited 35% and 21% overall retention in the mouse blood, respectively (FIG. 17A). The semilog plot of the retention-circulation time illustrates a bi-exponential decrease in RRBC particle concentration over time (FIG. 17A), indicating that the RRBC particle circulation follows a two-compartment pharmacokinetic model (Hu et al., 2011; Luk et al., 2014), where the particles first distribute from the plasma into various tissues, followed by a late (elimination) phase and the ultimate clearance from the blood. The elimination half-life of the RRBC particles was calculated as 41.8 h, which is similar to the values reported for most RBC membrane fused nanoparticles (Hu et al., 2011; Luk et al., 2014), indicating long in vivo circulation times which is potentially useful for cargo delivery functions. Following the pharmacokinetic examination, the biodistribution profile of the RRBC particles were assessed to confirm the potential of RRBC as an in vivo delivery vehicle. The mice were sacrificed at 2, 6, 24, and 48 hours post-injection of FITC-labeled RRBC particles and then the blood, liver, lung, kidney, spleen, and heart were harvested for fluorescence analysis. FIG. 17B shows the respective RRBC particle content per gram of tissue for all the organs examined. The majority of the particles were found to be localized in the liver and spleen 24 hours post-injection. This biodistribution pattern can be attributed to the erythrophagocytosis process, which removes senescent RBCs from the blood-stream and primarily takes place in the liver and spleen (Klei et al., 2017; Theurl et al., 2016). To further understand the overall RRBC particle distribution in each organ, the fluorescence signals were multiplied by the measured weight of the corresponding organ, assuming the weight of the blood as 7% of the total body weight. FIG. 17C shows the relative signal in each organ normalized to the total fluorescence. After accounting for the tissue mass, the RRBC particles were found to be mainly distributed in the blood and the liver. As the blood fluorescence decreased, a corresponding increase in fluorescence signal was observed in the liver, a highly perfused organ, attributed to the abundant blood flow through the liver, and the inescapable macrophage system uptake (Klei et al., 2017; Theurl et al., 2016). It is worth noting that, compared to the reported particle-circulation data in mouse models (Hu et al., 2011; Luk et al., 2014), for which most of the particles showed negligible blood retention after 24 hours, the RRBC particles displayed significantly longer circulation time, attributed to the sufficiently low modulus and the native RBC membrane camouflage, a feature which holds tremendous potential for biomedical applications as medicament delivery and bio-detection platforms.

The long circulation characteristics of the RRBC particle prompted us to assess its oxygen carrying capabilities since the most important function of the native RBCs is to transport oxygen from the lungs to the body tissues by virtue of hemoglobin (Hb) (Modery-Pawlowski et al., 2013; Jia et al., 2012; Duan et al., 2012). In order to achieve oxygen transport, hemoglobin was loaded within the polymer layer of the RRBC to yield an oxygen transport-capable material. The presence of hemoglobin was confirmed using luminol-based chemiluminescence (Doshi et al., 2009), a diagnostic tool commonly used in forensic science to detect blood stains (Hu et al., 2011; Luk et al., 2014) based on the iron-dependent catalysis of the reaction of the luminol-perborate with peroxide to produce a bluish glowing compound, which indicates the presence of iron-containing hemoglobin (Doshi et al., 2009). FIG. 17D shows that both the native RBCs and hemoglobin loaded RRBC particles could generate bluish glow when tested with luminol in the dark, demonstrating the presence of hemoglobin. To investigate the oxygen carrying capability of the hemoglobin-loaded RRBC particles, UV-Vis absorption analysis was used to reveal the reversible shift of the maximum absorption peaks of RBCs in oxygenated and deoxygenated states (Modery-Pawlowski et al., 2013; Jia et al., 2012; Duan et al., 2012). The characteristic absorption peak of the native RBC and hemoglobin-loaded RRBC particle in oxygenated states appeared at 415 nm (FIG. 17D) which after bubbling nitrogen gas for 2 hours and adding the reducing agent sodium dithionite (Na₂S₂O₄), red-shifted to 430 nm (FIG. 17D), confirming the deoxygenated states of the native RBCs and the RRBCs. These deoxygenated RRBCs could carry oxygen after exposure to atmospheric oxygen. This process of binding and release of oxygen could be repeated several times to show that the hemoglobin-loaded RRBCs had a similar oxygen carrying capability as the native RBCs (FIG. 17F). To further investigate the oxygen carrying capability of the RRBCs, the oxygenation rate of the deoxygenated form of RRBC was monitored by observing absorbance changes at 430 nm. Interestingly, both the native RBCs and the RRBCs displayed a similar oxygen curve profile (FIG. 17G), further confirming that hemoglobin-loaded RRBC has a similar oxygen carrying capability as the native RBCs.

Due to the demonstrated ability to load RRBC particles and the long term circulation seen in in vivo models, the loading of RRBC with various functional, non-native cargoes was assessed. RRBC particles were loaded with a magnetic resonance imaging (MRI) contrast agent, 5,10,15,20-Tetrakis(4-sulfonatophenyl)-21H,23H-porphine manganese(III) chloride (Mn-TPPS4, 3.5 μg/million particles) and the anti-cancer drug, doxorubicin hydrochloride (DOX, 5.5 μg/million particles). The evidence of loading and release profiles of these cargos were examined by dialyzing loaded RRBC particles in 1×PBS and then recording the absorbance (at 412 nm for Mn-TPPS4 and 485 nm for DOX) of the supernatant collected at different time points to determine the quantity of cargo released. Upon directly fusing the RBC membrane ghosts, the total Mn-TPPS4 released from the RRBC was calculated to be about 60% after 40 hours (FIG. 18D), which in the presence of an extra polymer layer between the loaded Mn-TPPS4 and the fused RBC ghost membrane, reduced to about 5% of Mn-TPPS4 (FIG. 18D). This significant decrease in the amount of Mn-TPPS4 released was attributed to an increase in the complexity of the diffusion pathway introduced by the extra polymer layer (FIG. 18A). The long-term retention of Mn-TPPS4 within the RRBC after polymer coating supports their in biomedical imaging applications (Liang et al., 2014). On the other hand, analysis of the DOX released from the RRBC revealed greater total DOX release at pH 5.0 (about 40%) compared to pH 7.4 (about 10%) after 40 hours (FIGS. 18A and 5E), possibly due to the carboxylate groups on the alginate layer becoming less negatively charged at lower pH, and reducing the attractive electrostatic interactions between the alginate layer and the DOX molecules, which should lead to an increased rate of DOX release (Gao et al., 2017; Li et al., 2016). This pH dependent and slow release suggests a possible lower toxicity of DOX to normal tissues at physiological conditions versus potentially greater anti-cancer activity of the DOX loaded RRBC particles in the acidic tumor micro-environment due to higher DOX release (FIG. 18B). Besides small molecules, evidence of nanoparticle cargo loading onto RRBCs was demonstrated. To prove this, magnetically active RRBCs were fabricated by packaging RRBC with magnetic iron oxide (Fe₃O₄) nanoparticles. By controlling the external magnetic field, the ‘magnetic RRBC’ exhibited an ‘on’ and ‘off’ magnetic guidance/movements (FIGS. 18C and 18H), that are also potentially relevant for magnetic drug targeting, toxin sequestration (FIGS. 18I and 18J), magnetic resonance imaging (MRI), magnetic hyperthermia, or the development of micromotors (Wu et al., 2015; Wu et al., 2014; Espinosa et al., 2016; Yao et al., 2017).

To further demonstrate the multifunctionality of RRBCs, the possible use of RRBCs as a platform for detoxification and biosensing was examined. Globally, bacterial infections are the leading cause of morbidity and mortality worldwide (Hu et al., 2013; Wei et al., 2017; Chen et al., 2018; de Avila et al., 2018). Many of these bacteria release pore-forming toxins (PFTs) or cytotoxic proteins which are major factors in the virulence of these bacteria. PFTs form transmembrane pores on the cellular membrane, allowing uncontrolled transport of solutes across the membrane, leading to cell death via colloid-osmotic lysis (Hu et al., 2013; Wei et al., 2017; Chen et al., 2018; de Avila et al., 2018). In the design of the RRBC, the outer RBC-derived membrane can provide an ideal surface to absorb and neutralize PFTs, supported by the inner polymer core's ability to stabilize the membrane and prevent PFT desorption. (FIG. 18I) To investigate the PFT neutralization by the RRBC particles, a classical PFT, α-hemolysin (Hlα) was pre-incubated with equivalent amounts of RBC ghost membranes, RBC-polymer replicas, or RRBCs, and then mixed with 4-times equivalent of purified RBCs before hemolysis tests. Almost zero hemolytic effect was observed when Hlα was pre-incubated with the RRBC particles, relative to about 100% or about 90% RBC hemolysis from Hlα pre-incubated with the RBC ghost membrane or the RBC-polymer replica respectively, indicating that only polymer-stabilized RBC membrane in the RRBCs could sequester Hlα (during pre-incubation) and prevent RBC hemolysis (FIGS. 18J and 26). To further investigate the PFT neutralization activity, RBC-polymer replicas or RRBCs were mixed with the purified native RBCs (1:4 ratio), and then Hlα was added so that either of the constructs could compete with the purified RBCs for the Hlα toxin. In this ‘competitive’ assay, only about 50% RBC hemolysis was observed with the RRBCs compared with about 90% hemolysis in the case of the RBC-polymer replica (FIG. 18J), indicating that the RRBC particles effectively compete for and sequester Hlα toxins and suggesting their use as decontamination agents.

Under normal physiological conditions, the native RBCs are characterized by high levels of adenosine triphosphate (ATP) that enable them to perform requisite physiological functions (Sikora et al., 2014; Meli et al., 2018). Therefore, measurement of the release of ATP due to RBC hemolysis, represents a possible way of detecting the presence of toxins that trigger RBC hemolysis (FIG. 18I). In order test this concept, a luciferase-luciferin ATP biosensor was loaded within either RBC-polymer replicas or RRBCs that were then mixed with intact RBCs in PBS, lysed RBCs in PBS, or lysed RBCs in water followed by luminescence testing. Interestingly, of these systems, intact RBCs yielded no response, RBC-polymer replicas yielded a luminescent response for RBCs lysed in either PBS or water, but the biosensor loaded RRBCs yielded a luminescent response only when incubated with RBCs lysed in water (FIG. 18K). This is explained by the fact that the biosensor loaded within the polymer replica is accessible to ATP in both water and PBS. However, the biosensor encapsulated beneath the RBC membrane in the RRBC is inaccessible in PBS and accessible only in water that osmotically lyses/permeabilizes the RRBC membrane. This suggests that the RRBC biosensor should be stable under normal physiological conditions but be activated only under conditions where RBCs are lysed producing ATP and the RRBC membrane is permeabilized. These conditions are met under conditions where PFTs (e.g., Hlα) induce RBC lysis and permeabilization of the RRBC membrane. Due to the luminescence intensity of luciferase-luciferin ATP biosensor loaded RRBCs increases linearly with increasing hemolysis content of RBCs lysis water solution, the luminescence intensity-hemolysis standard curve was determined (FIG. 26). To evaluate applications of the RRBC biosensor based on ATP release, luciferase-luciferin ATP biosensor loaded RBC-polymer replicas or RRBCs were first pre-incubated with Hlα, and then mixed with the native RBCs (1:4 ratios). Notably, after pre-incubation, biosensor loaded RRBCs exhibited very low luminescence signal (about 0% hemolysis), the biosensor loaded RBC-polymer replicas exhibited a 28-fold higher luminescence signal (about 90% hemolysis) compared to RRBCs (FIG. 18K), which is consistent with PFT neutralization results (FIG. 18J). To further mimic the typical competitive binding in the body, biosensor loaded RBC-polymer replicas or RRBCs were mixed with the native RBCs (1:4 ratios) and then Hlα was added. A lower luminescence intensity was observed with the biosensor loaded RRBCs (about 50% hemolysis) relative to that of the biosensor loaded RBC polymer replicas (about 90% hemolysis), which matched the Hlα's induced hemolysis observed in direct competitive setup. This study demonstrates the potential of our designed biosensor loaded RRBCs as sensors for monitoring pore-forming toxin in vivo system.

In summary, a pathway is presented to construction synthetic RRBCs that mimic the unique characteristics of native RBCs and expand upon them to provide novel functionalities. The RRBC particles were constructed by three stages of assembly: silica cell bio-replication of native RBCs, layer-by-layer deposition of a biocompatible polymer to translate the native RBC shape into a flexible RBC-polymer replica loadable with functional cargos, and encapsulation of the functionalized RBC-polymer within a RBC derived membrane ghost that confers to the RRBC the surface properties of the native RBCs. The RRBC particle displays deformability typical of native RBCs, zero hemolytic activity, low cytotoxicity and sustained vascular flow in the ex-ovo chick embryo and in in vivo mouse models. In addition, different functional cargos (e.g., hemoglobin, Mn-TPPS4, DOX, iron oxide nanoparticles and ATP biosensor) can be loaded onto the RRBC particle to enable functions such as oxygen delivery capability, MRI contrast imaging, therapeutic drug delivery, magnetic field directed movement and localization, and pore-forming toxin biosensing. Together, RRBCs have been engineered with robust and unique features that may enablethem to serve as a tool to promote understanding of complex life processes and possibly as a new multifunctional delivery and bio-detection platform.

Red blood cells (RBCs) possess unique characteristics relative to other cells, making them attractive targets for cellular biomimicry. Here micron size multifunctional rebuilt RBC particle is designed and constructed to be used as therapeutic delivery and bio-detection platforms, as well as serve as a unique tool to advance our understanding of complex life processes.

Materials

All chemicals and reagents were used as received. Tetramethyl orthosilicate (99%, TMOS), sodium chloride (NaCl), hydrochloric acid (37%, HCl), Fluorescein-5-isothiocyanate (FITC), chitosan, alginate, formaldehyde (37%), glutaraldehyde solution (25% in H₂O), endothelial cell growth medium, dimethyl sulfoxide (DMSO), sodium perborate, sodium carbonate, luminol, sodium dithionite (Na₂S₂O₄), hemoglobin, doxorubicin (DOX), Mn(III)tetra (4-sulfonatophenyl) porphyrin, Iron(III) acetylacetonate [Fe(acac)₃], benzyl alcohol, α-hemolysin, luciferase and luciferin were purchased from Sigma-Aldrich. 1×-phosphate-buffered saline (1×PBS) and Blood Typing Anti-Sera, Anti-A, Anti-B and Anti Rh were purchased from Thermo Fisher Scientific. Buffered Oxide Etch (BOE) was purchased from KMG Chemicals. 1,2-dioleoyl-sn-glycero-3-phosphoethanolamine-N-(lissamine rhodamine B sulfonyl) (18:1 Liss Rhod PE) lipid was purchased from Avanti Lipids. Ethanol was purchased from KOPTEC. Human umbilical vein endothelium cells (HUVEC) and mouse macrophage Raw264.7 cells were obtained from the American Type Culture Collection (ATCC). Dulbecco's modification of Eagle's medium (DMEM) was obtained from Corning Cellgro. Fluorescein labeled Lens Culinaris Agglutinin (LCA) was purchased from Vector Laboratories. Milli-Q water with a resistivity of 18.2 MO cm was obtained from an inline Millipore RiOs/Origin water purification system.

Purification of RBCs

All the animal procedures complied with the guidelines of the University of New Mexico Institutional Animal Care and Use Committee and were conducted following institutional approval (Protocol 11-100652-T-HSC and 17-200658-HSC). Human RBCs were acquired from healthy donors with their informed consent. All blood samples were collected and stored in BD Vacutainer® blood collection tubes (Becton Dickinson, N.J., USA) containing 1.5 mg of EDTA per mL of blood for anticoagulation purposes. The purification of whole blood was carried out using Ficoll® density gradient centrifugation procedure.

Preparation of Silica RBC Replicas

Purified RBCs were fixed in 4% formaldehyde in 1×PBS at room temperature for 20 h before silicification. The fixed RBCs were rinsed twice with 1×PBS, once with 154 mM NaCl solution (0.9% saline) and then suspended in a silicification solution containing 100 mM TMOS, 154 mM NaCl and 1.0 mM HCl (pH 3.0). After 24 h rotation at room temperature to allow silicification process to take place, silicified RBCs were subjected to series of ethanol dehydration (30, 50, 70, 90, 100% ethanol in water) for 10 minutes each and then dried under vacuum for 24 h. Dry silicified RBCs were then calcined at 500° C. for 4 h in an oven by placing them in a covered (but not airtight) glass tube to generate silica RBC replicas.

Preparation of RBC-Polymer Replica

The silica RBC replicas were incubated for 2 h in chitosan solution (2 mg/mL in 1% acetic acid solution) under constant shaking. After rinsing with water, the particles were resuspended in alginate solution (1 mg/mL in water) under constant shaking for 0.5 h. Then, the particles were rinsed with water and isolated via centrifugation (1500 g for 5 minutes). This process represents the typical procedure for single chitosan-alginate layer formation and it was repeated two times to achieve polymer coated silica RBC replica. In order to fabricate RBC-polymer replica, 1:10 diluted buffered oxide etch, also known as buffered HF (BOE) solution (pH-5) was used to etch the silica and yield RBC-polymer replica. The RBC-polymer replica were washed with water and resuspended in double distilled water.

Preparation of RBC-Membrane-Derived Ghosts

Purified RBCs were washed three times with ice cold 1×PBS, and then suspended in ice cold 0.25×PBS for 20 min to allow hemolysis to take place. After treatment with hypotonic solution (0.25×PBS), the released hemoglobin was removed via centrifugation (1000 g for 5 minutes), whereas the pellet (RBC ghost) with light pink color was collected and washed twice with 1×PBS. The RBC ghosts (devoid of cytoplasmic contents) were mixed with 1,2-dioleoyl-sn-glycero-3-phosphoethanolamine-N-(lissamine rhodamine B sulfonyl) (18:1 Liss Rhod PE) and verified under fluorescence microscope, which revealed a hollow spherical structure of RBC membrane ghosts.

Preparation of Rebuilt RBC (RRBC) Particles

To prepare the RRBC particles, the RBC membrane ghosts were fused onto RBC-polymer replica. Briefly, 4×10⁷ RBC-polymer replica quantified by hemocytometer were mixed with RBC membrane ghosts prepared from 1×10⁸ purified RBCs and then sonicated in ice cold water bath for 1 min. An excess of RBCs was used to compensate for the membrane loss during RBC ghost derivation. The resulting RRBC particles were carefully centrifuged (5000 g for 10 minutes) and the excess membrane components remaining in the supernatant were removed.

Zeta Potential Measurements

Zeta potential measurements were made using Malvern Zetasizer Nano-ZS (Westborough, Mass., USA) equipped with a He—Ne laser (633 nm) and non-invasive backscatter optics (NIBS). The layer-by-layer samples, silica RBC replica, RBC-polymer replica, and RRBC particles for zeta potential measurements were suspended in 5 mM NaCl solution, while the zeta potential measurements for the RBC and RRBC particles was acquired in 154 mM NaCl solution (0.9% saline) using mono modal analysis tool. All reported values correspond to the average of at least three independent samples.

Scanning Electron Microscopy (SEM) Imaging

The morphology of fixed RBC, silica RBC replica, polymer coated silica RBC replica and RBC-polymer replica samples were characterized using scanning electron microscope (SEM). SEM samples were prepared by drop casting. Briefly, all samples were suspended in water, and then dropped onto 5×5 mm glass slides. The glass slides were then mounted on SEM stubs using conductive adhesive tape (12 mm OD PELCO Tabs). Samples were sputter coated with a 10 nm layer of gold using a Plasma Sciences CrC-150 Sputtering System (Torr International, Inc.). SEM images were acquired under high vacuum, at 10 kV, using an FEI Quanta series scanning electron microscope (Thermo Fisher Scientific, MA, USA).

Confocal Microscopy Imaging

RRBC particles were spotted onto glass cover slips. Slides were mounted using Vectashield Antifade. Confocal images were acquired with a 63×/1.4NA oil objective in sequential scanning mode using a Leica TCS SP8 confocal microscope.

Microfluidic Blood Capillary Model and Experiments

The microfluidic blood capillary model mimicking dimensions (5 μm in diameter and 50 μm in length) and pressure drops of human blood capillaries was prepared as described previously (Ciu et al., 2014; Sun et al., 2015). In brief, a master pattern was designed using computer-aided design software (AutoCAD 2013, Autodesk, USA) and then simulation software (COMSOL Multiphysics 4.3, USA) was used to refine and validate the design. The designed master pattern was then transferred to the silicon wafer using chrome mask and negative photoresist and then transferred into polydimethylsiloxane (PDMS) to achieve microfluidic blood capillary device through soft lithography. The microfluidic experiments were then performed as follows. The non-sample reservoirs were filled with 1×PBS, and the device was then connected to a pressure controller (NE-300, New Era Pump System) and placed on top of a Zeiss AxioExaminer upright microscope. For the microfluidic tests, 1×10⁶/mL particles (RBCs, silica RBC replicas, and RRBC particles cross-linked with different cross-linker concentrations) in PBS were assessed for deformability characteristics. The different pressures used for the two inlets were chosen based on the finite element simulations to achieve physiologically relevant pressure drops over the microchannels (see Table 2).

TABLE 2
Simulation of flow in the microfluidic blood capillary model

| Average pressure | Flow rate at high | Flow rate at low |
|---|---|---|
| drop across central | pressure outlet, | pressure outlet, |
| microchannels, mbar | μL min⁻¹ | μL min⁻¹ |
| 2.2 | 18 | 10 |
| 6.7 | 34 | 12 |
| 20 | 79 | 16 |

Antibody-Mediated Agglutination Assay

Briefly, 1×10⁶ native RBCs or RRBC particles were suspended in 450 μL of 1×PBS (pH 7.4) solution, and then 50 μL of anti-type sera [anti-A, anti-B, and anti-D (Rh)] were added. After 15 min, the bright field images were acquired on the Leica DM13000 B inverted microscope to evaluate occurrence of agglutination or lack thereof.

Immunofluorescence Staining

The native RBC and RRBC particles were blocked with 5% BSA in 1×PBS, and then incubated with fluorescent antibodies against ICAM-4 (R&D Systems) and CD47 (Biolegend) proteins for 30 min. The samples were then rinsed with 1×PBS, and then suspended again in 1×PBS. Microscopy images were then obtained on the Leica DM13000 B inverted fluorescence microscope.

Hemolysis Assay

Purified RBCs were incubated with different concentrations of silica RBC replicas, polymer coated silica RBC replicas, RBC-polymer replica, and RRBC particles at 37° C. for 2 h in continuous rotating state. Double distilled (D.I.) water and 1×PBS containing purified RBCs were used as the positive and negative controls, respectively. The absorbance of hemoglobin in the supernatant was measured by a BioTek microplate reader (Winooski, Vt.) at 540 nm. The hemolysis percentage of each sample was determined using the reported equation (Townson et al., 2013; Durfee et al., 2016) as; Percent hemolysis (%)=100*(Sample Abs_{540 nm}−Negative control Abs_{540 nm})/(Positive control Abs_{540 nm}−Negative control Abs_{540 nm})

Cell Viability Assay

Cell culture was performed using standard procedures. HUVEC and Raw264.7 cells were maintained in the respective media of endothelial cell growth medium and DMEM containing 10% FBS at 37° C. and 5% CO₂. Cells were passaged at approximately 70% confluency. For cell viability assays, 100 μL of cell suspension (100,000 cells/mL) were seeded into a 96-well plate (White Opaque) and cultured for 24 h at 37° C. The cells were then incubated with 100 μL of different concentrations of silica RBC replicas, polymer coated silica RBC replicas, RBC-polymer replica, and RRBC particles solutions. After 24 h incubation, 100 μL of CellTiter-Glo 2.0 Reagent was added into each well and incubated for 10 min at room temperature. The luminescence readings were then obtained/recorded using BioTek microplate reader. The percent cell viability was calculated relative to the control non-treated cells.

Test of Vascular Flow in Ex Ovo Chick Embryos

The vascular flow characteristics of RRBC particles were tested using Ex ovo chick embryo model as described previously (Townson et al., 2013) and was conducted following institutional approval (Protocol 11-100652-T-HSC). Briefly, eggs were acquired from East Mountain Hatchery (Edgewood, N. Mex.) and placed in a GQF 1500 Digital Professional incubator (Savannah, Ga.) for 3-4 days. Embryos were then removed from shells by cracking into 100 ml polystyrene weigh boats. Ex ovo chick embryos were covered and incubated at 37° C., 70% humidity. 50 μL (at 4×10⁷ particles/mL) of samples (RBC-polymer replica, silica RBC replica©polymer-RBC ghost [silica-RRBC], and RRBC particles) in 1×PBS were injected into the secondary ortertiary veins via pulled glass capillary needles and then, the CAM vasculature was imaged using a customized avian embryo chamber and a Zeiss Axio Examiner upright microscope with heated stage.

Pharmacokinetics and Biodistribution Studies

All the animal procedures complied with the guidelines of the University of New Mexico Institutional Animal Care and Use Committee and were conducted following institutional approval (Protocol 17-200658-HSC). The experiments were performed on female Albino C57BL/6 mice (6 weeks) from Charles River Laboratories. To evaluate the circulation half-life of RRBC particles, 100 μL of FITC-labeled RRBC particles (3×10⁷ particles/mL) were administered by retro-orbital injections. Blood was collected at 2, 6, 24, and 48 h post-injection with each group contained three mice. Blood samples were diluted 1:1 with PBS prior to fluorescence measurements. Particle retention in circulation was determined by measuring the fluorescence using a BioTek microplate reader (Winooski, Vt.). Pharmacokinetics parameters were calculated to fit a two-compartment model. To calculate the elimination half-life, the normalized signal was transformed using natural log, and the elimination phase from the resulting data was fitted using a linear regression curve. Elimination half-life was calculated as t_{1/2}=ln(2)/8, where 13 is the negative slope obtained from the fit. The value of 13 with standard error for the RRBC particles was 0.01655±0.0028.

To study the biodistribution of the RRBC particles in various tissues, similar to the preceding study, 100 μL of FITC-labeled RRBC particles were administered by retro-orbital injection. At 2, 6, 24, and 48 h following the particle injection, three mice were randomly selected and euthanized. Their blood, liver, lung, kidney, spleen, and heart were collected. The collected organs were weighed and then homogenized in 1 mL PBS. Total weight of blood was estimated as 7% of mouse body weight. The fluorescence intensity of each sample was determined by a BioTek microplate reader (Winooski, Vt.).

Hemoglobin Loading

The chitosan surface of the RBC-polymer replica was incubated for 24 h with 5 mg/mL hemoglobin under constant shaking at 4° C. After rinsing with water, the particles were resuspended in 2 mg/mL chitosan solution under constant shaking for 0.5 h at 4° C. This process was repeated three times to achieve enough hemoglobin loading. The particles were then resuspended in 1 mg/mL alginate solution under constant shaking for 0.5 h and then the RBC membrane ghosts were fused onto hemoglobin loaded RBC-polymer replica to generate hemoglobin loaded RRBC particles.

Chemiluminescence Assays

The luminol-based method for evaluating oxygen carrying ability of the RRBC particles was adopted from Doshi, Nishit, et al. (Doshi et al., 2009). Briefly, 70 mg sodium perborate, 500 mg sodium carbonate, and 200 mg luminol were added to 5 mL water and dissolved with sonication to achieve luminol solution. The luminol solution was left undisturbed for 5 min in a dark room. For imaging purposes, 1 mL of luminol solution was added to 4 mL samples (5 million native RBCs and RRBC particles) in 1×PBS (pH 7.4) solution. The optical image was taken by Sony ILCE-5100 Camera (ISO-100 and exposure time 1/15s). The chemiluminescence optical image was taken in a dark room by Sony ILCE-5100 Camera (ISO-6400 and exposure time 30 s). For luminescence assay, 100 μL of samples (5 million native RBCs and RRBC particles) in 1×PBS (pH 7.4) solution were added into white 96-well plates at a density of 5 million cells/mL. After that, 20 μL of luminol solution was added to each well. Mix the contents for 2 min on shaker in the dark. Luminescence was measured using a BioTek microplate reader. The luminescence was expressed as a relative percentage of the control.

Assay of the Reversible Binding of Oxygen

The ability of the RRBC particle to reversibly binding oxygen was detected by analyzing changes of UV-Vis absorption spectrum (300-700 nm) in oxygenated and deoxygenated solutions (Jia et al., 2012; Duan et al., 2012) For complete deoxygenation, nitrogen gas was bubbled into the sample solution to displace oxygen. After 2 h, sodium dithionite (Na₂S₂O₄) was added, and UV-Vis absorption spectrum was obtained by a BioTek microplate reader. For oxygenation, sample solutions were exposed to atmospheric oxygen for more than 2 h, and then UV-Vis absorption spectrum was recorded as before. This process represents the typical procedure used to test reversible oxygen binding capability and it was repeated two times. The deoxygenated sample (λₘₐₓ=430 nm) could be gradually converted to oxygenated sample (λₘₐₓ=415 nm) by exposing it to air atmosphere at room temperature. The oxygenation rate of the deoxygenated sample was monitored by observing changes in absorbance via UV-Vis spectroscopy. The oxygenation state of each sample was calculated using the following equation: Oxygenation state (%)=100*(Absₜ₀−Absₜ)/(Absₜ₀−Absₜₘᵢₙ) where Absₜ₀ and Absₜ represent the 430 nm absorbance at the starting point (t=0, complete deoxygenated state) and at the specific time, respectively, and Absₜₘᵢₙ represents the 430 nm absorbance at the minimum value.

Loading and Release Kinetics of Small Molecules

In order to load the negatively charged Mn-TPPS4, the chitosan surface of the RBC-polymer replica was incubated with Mn-TPPS4 (2 mg/mL) for 4 h under constant shaking. RBC membrane ghosts were then directly fused onto the Mn-TPPS4 loaded RBC-polymer replica. Note that for the extra polymer layer samples, the particles were resuspended in 1 mg/mL alginate solution under constant shaking for 0.5 h and then the RBC membrane ghosts were fused onto the Mn-TPPS4 loaded RBC-polymer replica. For the positively charged doxorubicin (DOX) loading, the alginate surface of the RBC-polymer replica was incubated for 4 h in 3 mg/mL DOX under constant shaking. Then, the particles were resuspended in 1 mg/mL alginate solution under constant shaking for 5 min followed by the fusion of the RBC membrane ghosts onto the DOX loaded RBC-polymer replica. To quantify loading of Mn-TPPS4 and DOX, microplate reader UV-Vis measurements were obtained at 410 nm for Mn-TPPS4 and 475 nm for DOX. The Mn-TPPS4 loading capacity was found to be 3.5 μg/million particles while the DOX loading capacity was 5.5 μg/million particles. The dialysis bag diffusion method was used to evaluate Mn-TPPS4 and DOX release kinetics. Briefly, particles were loaded into 20 kDa MWCO Por Float-A-Lyzer G2 dialysis device, sealed in 50 mL conical tubes containing 20 mL phosphate-buffered saline (pH 7.4 or 5), and kept at 37° C. while stirring. At definite time points, 1 mL of dialysate was removed for absorbance analysis on a BioTek microplate reader and then 1 mL of the fresh dialysate solution was added to the conical tube. Each batch of experiments was performed in triplicate.

Magnetic Iron Oxide Nanoparticles Synthesis

Bare magnetic iron oxide (Fe₃O₄) nanoparticles were synthesized according to the previous method (Li et al., 2016). Briefly, 0.687 g of Fe(acac)₃ (1.94 mmol) was dissolved in 9 mL of benzyl alcohol. The solution was heated to 170° C. under reflux and stirring at 1500 rpm for 24 h. After the reaction was cooled down to room temperature, 35 mL of EtOH was added into the mixture, and then centrifuged at 20000 rpm for 10 min. The supernatant was discarded, and the resulting precipitate was washed with EtOH twice to yield the required Fe₃O₄ nanoparticles. The synthesized Fe₃O₄ nanoparticles were stored in EtOH before use.

Loading of Magnetic Nanoparticles

The bare Fe₃O₄ nanoparticles (NP) were incubated in 1 mg/mL chitosan solution overnight and then washed with DI water twice prior to the actual loading experiments. Chitosan coated Fe₃O₄ nanoparticles were then incubated with the alginate surface of the RBC-polymer replica for several hours under constant shaking followed by another resuspension in alginate solution (1 mg/mL) under constant shaking for 0.5 h to ensure optimal display of the alginate's negative charge. The RBC membrane ghosts were then fused onto the Fe₃O₄ loaded polyion to generate Fe₃O₄ loaded RRBC particles.

Confirmation of the Fe3O4 Nanoparticle Loading

The magnetic Fe₃O₄ nanoparticle loaded RRBC particles were suspended in an external magnetic field produced by a neodymium magnet. The bright field images were then obtained on the Leica DMI3000 B inverted microscope to evaluate the magnetic guidance response.

Quantification of Toxin Hemolytic Activity

Briefly, 1.5×10⁷ purified RBCs were incubated with 1 mL of different concentrations of α-hemolysin in PBS at 37° C. for 30 min. D.I. water and 1×PBS containing purified RBCs were used as the positive and negative controls, respectively. The absorbance of hemoglobin in the supernatant was measured by a BioTek microplate reader (Winooski, Vt.) at 540 nm. The hemolysis percentage of each sample was determined using the reported equation (Townson et al., 2013; Durfee 2016) as; Percent hemolysis (%)=100*(Sample Abs_{540 nm}−Negative control Abs_{540 nm})/(Positive control Abs_{540 nm}−Negative control Abs_{540 nm})

Toxin Absorption Study In Vitro

Briefly, 3×10⁶ RRBC particles were incubated with 1 mL of different concentrations of α-hemolysin in PBS at 37° C. for 30 min., followed by addition of 1.2×10⁷ purified RBCs for additional 30 min incubation. The hemolysis percentage of each sample was determined using the absorbance of hemoglobin in the supernatant.

Toxin Neutralization In Vitro

In vitro toxin neutralization ability of RRBC particles was examined in two parts. In part 1, 1 mLα-hemolysin (20 μg/mL in PBS) was incubated with RBC ghost membrane (from 7.5×10° RBCs), 3×10° RBC polymer replicas, and 3×10° RRBC particles at 37° C. for 30 mins. After this incubation, 1.2×10⁷ purified RBCs were added into the respective formulations. The mixture was incubated for an additional 30 min at 37° C. prior to hemolysis quantification. In part 2, 1 mLα-hemolysin (20 μg/mL in PBS) was directly incubated with the mixture of 3×10° RRBC particles and 1.2×10⁷ purified RBCs at 37° C. for 30 mins, followed by hemolysis quantification. The hemolysis percentage of each sample was determined using the absorbance of hemoglobin in the supernatant.

Luciferase-Luciferin Biosensor Loading

The alginate surface of the RBC-polymer replica was incubated for 24 h with 3 mg/mL luciferin under constant shaking in the dark. After rinsing with water, the particles were resuspended in 1 mg/mL alginate solution for 10 min, and then incubated in 2 mg/mL chitosan solution for 30 min. For the luciferase loading, the chitosan surface of the luciferin loaded RBC-polymer replica was incubated overnight in 1 mg/mL luciferase in PBS under constant shaking at 4° C. in the dark. Then, the particles were resuspended in 2 mg/mL chitosan solution under constant shaking for 5 min, followed by 1 mg/mL alginate solution for 10 min. In order to have more luciferin, 3 mg/mL luciferin was added to luciferase-luciferin loaded RBC-polymer replica again and incubated for 4 h under constant shaking at 4° C. in the dark. After rinsing with PBS, the particles were then resuspended in 1 mg/mL alginate solution under constant shaking for 5 min and then the RBC membrane ghosts were fused onto luciferase-luciferin biosensor loaded RBC-polymer replica to generate luciferase-luciferin biosensor loaded RRBC particles.

Luciferase Activity Assay

Since the goal was to test the ATP released from the RBCs, the biological activity of luciferase was measured using different concentrations of water lysed RBC solution. In brief, a solution containing 1.2×10⁶, 3×10⁶, 6×10⁶, 9×10⁶, and 1.2×10⁷ lysed RBCs in water (10%, 25%, 50%, 75%, 100% hemolysis, respectively) was added to a solution of 3×10⁶luciferase-luciferin biosensor loaded RRBC particles and MgSO4 (8 mM) in a final volume of 1 mL. Luminescence intensity was then measured immediately by a BioTek microplate reader (Winooski, Vt.).

Evaluation of Pore-Forming Toxin Sensor

The Pore-forming toxin sensor property of RRBC particles was assessed in three parts. In the first part, 1 mL PBS or D.I. water, and toxin (20 μg/mL in PBS) was incubated with a mixture of 3×10⁶ luciferase-luciferin biosensor loaded RRBC particles or luciferase-luciferin biosensor loaded RBC polymer replicas and 1.2×10⁷ purified RBCs at 37° C. for 15 mins, followed by luminescence measurements. In the second part, 1 mL of 1.2×10⁷ lysed RBCs in PBS was incubated with 3×10⁶ luciferase-luciferin biosensor loaded RRBC particles or luciferase-luciferin biosensor loaded RBC polymer replicas at 37° C. for 15 mins, respectively, followed by luminescence measurements. These lysed RBCs in PBS were generated through bath sonicated disruption of RBCs. In the third part, 1 mL α-hemolysin (20 μg/mL in PBS) was incubated with 3×10⁶ luciferase-luciferin biosensor loaded RRBC particles or luciferase-luciferin biosensor loaded RBC polymer replicas at 37° C. for 15 mins, and then 1.2×10⁷ purified RBCs were added into the respective formulations. The mixture was incubated for an additional 15 min at 37° C. prior to luminescence quantification. Luminescence intensity was measured immediately by a BioTek microplate reader (Winooski, Vt.).

### Example 3

In one embodiment, a method to prepare a red blood cell mimetic includes silificating and calcinating vertebrate red blood cells, thereby providing silica replicated vertebrate red blood cells; coating the silica replicated vertebrate red blood cells with one or more flexible polyion polymers in one or more layers, thereby yielding coated silica replicates red blood cells; desilifying, e.g., etching, the coated silica replicated red blood cells, thereby yielding red blood cell-polymer replicas; and fusing the red blood cell-polymer replicas with lipid bilayers. In one embodiment, the vertebrate red blood cells are fixed. In one embodiment, the red blood cells are primate cells. In one embodiment, the red blood cells are human red blood cells. In one embodiment, the lipid layers are from hematopoietic cells other than red blood cells. In one embodiment, the lipid layers are from non-hematopoietic cells. In one embodiment, the lipid layers are from red blood cells. In one embodiment, the lipid layers are functionalized with one or more molecules, e.g., before, during or after the lipid layers are contacted with the desilified, coated silica replicated red blood cells. In one embodiment, the desilified, coated silica replicated red blood cells are functionalized with one or more molecules. In one embodiment, at least one of the molecules is a contrast agent, e.g., MRI contrast agent. In one embodiment, at least one of the molecules is a substrate for an enzyme. In one embodiment, at least one of the molecules is a chemotherapeutic agent. In one embodiment, at least one of the molecules is a nanoparticle. In one embodiment, the lipid bilayers are membranes from vertebrate red blood cell ghosts. In one embodiment, the silica replicated red blood cells are coated with two or more distinct polyionic polymers. In one embodiment, the coat has alternating layers of the two or more distinct polyionic polymers. In one embodiment, one polymer is a polycationic polymer and the other polymer is a polyanionic polymer. In one embodiment, the polyion comprises a polysaccharide, chitosan, polyarginine, or polylysine. In one embodiment, the polyion comprises alginate, pectin, xanthan gum, carboxymethyl cellulose, dextran sulfate, agar, or carrageenan. In one embodiment,

the red blood cells prior to silificating are fixed with formaldehyde, acrolein, glyoxal, osmium tetroxide, carbodiimide, diimidoester, chloro-s-traizide, diisocyanate, ethanol, picric acid, or glutaraldehyde. In one embodiment, the one or more molecules comprise nucleic acid, protein, peptides, antibodies, drugs, or hemoglobin. In one embodiment, the one or more molecules comprise an anti-cancer agent, an anti-viral agent, an anti-bacterial agent, or an anti-parasite agent. In one embodiment, the one or more molecules
comprise a cytokine or growth factor. In one embodiment, the one or more molecules comprise a MRI contrast agent. In one embodiment, the desilifying comprises etching.

In one embodiment, silica replicated vertebrate red blood cells are coated with one or more flexible polyion polymers in one or more layers, thereby yielding coated silica replicates red blood cells; the coated silica replicated red blood cells are etched, thereby yielding red blood cell-polymer replicas; and the red blood cell-polymer replicas with lipid bilayers are fused with lipid bilayers, e.g., lipid bilayers from cells such as red blood cells. In one embodiment, the vertebrate red blood cells are fixed. In one embodiment, the red blood cells are primate cells. In one embodiment, the red blood cells are human red blood cells. In one embodiment, the lipid layers are from hematopoietic cells other than red blood cells. In one embodiment, the lipid layers are from non-hematopoietic cells. In one embodiment, the lipid layers are from red blood cells. In one embodiment, the lipid layers are functionalized with one or more molecules, e.g., before, during or after the lipid layers are contacted with the desilified, coated silica replicated red blood cells. In one embodiment, the desilified, coated silica replicated red blood cells are functionalized with one or more molecules. In one embodiment, at least one of the molecules is a contrast agent, e.g., MRI contrast agent. In one embodiment, at least one of the molecules is a substrate for an enzyme. In one embodiment, at least one of the molecules is a chemotherapeutic agent. In one embodiment, at least one of the molecules is a nanoparticle. In one embodiment, the lipid bilayers are membranes from vertebrate red blood cell ghosts. In one embodiment, the silica replicated red blood cells are coated with two or more distinct polyionic polymers. In one embodiment, the coat has alternating layers of the two or more distinct polyionic polymers. In one embodiment, one polymer is a polycationic polymer and the other polymer is a polyanionic polymer. In one embodiment, the polyion comprises a polysaccharide, chitosan, polyarginine, or polylysine. In one embodiment, the polyion comprises alginate, pectin, xanthan gum, carboxymethyl cellulose, dextran sulfate, agar, or carrageenan. In one embodiment,

the red blood cells prior to silificating are fixed with formaldehyde, acrolein, glyoxal, osmium tetroxide, carbodiimide, diimidoester, chloro-s-traizide, diisocyanate, ethanol, picric acid, or glutaraldehyde. In one embodiment, the one or more molecules comprise nucleic acid, protein, peptides, antibodies, drugs, or hemoglobin. In one embodiment, the one or more molecules comprise an anti-cancer agent, an anti-viral agent, an anti-bacterial agent, or an anti-parasite agent. In one embodiment, the one or more molecules
comprise a cytokine or growth factor. In one embodiment, the one or more molecules comprise a MRI contrast agent. In one embodiment, the desilifying comprises etching.

In one embodiment, a red blood cell mimetic comprises round and biconcave etched silica having one or more polyion layers surrounded by a lipid bilayer. In one embodiment, one of the layers comprises chitosan. In one embodiment, one of the layers comprises alginate. In one embodiment, the coat comprises alternating layers of chitosan and alginate. In one embodiment, one or more selected molecules are between the outermost polyion layer and the lipid bilayer. In one embodiment, one or more selected molecules are between polyion layers. In one embodiment, the lipid bilayer is functionalized with one or more molecules, e.g., via a covalent linkage. In one embodiment, the lipid bilayer is functionalized before fusion.

### REFERENCES

Bochev et al., Journal of Biochemical and Biophysical Methods 27:301(1993).
Chen et al., Advanced Healthcare Mater., 1701366 (2018).
Cohen et al., Blood 114:3668 (2009).
Cui et al., Adv. Materials 26:7295 (2014).
de Avila et al., Sci. Robot. 3:0485 (2018)
de Back et al., Frontiers Physiology 5:9 (2014).
Doshi et al., Proc. Natl. Acad. Sci. 106:21495 (2009).
Duan et al., ACS Nano 6:6897 (2012).
Durfee et al., ACS Nano. 10:8325 (2016).
Espinosa et al., ACS Nano 10:2436 (2016).
Fang et al., Nanoscale 5:8884 (2013).
Gao, et al., Nanoscale 9:12533 (2017).
Hu et al., Nature Nanotechnology 8.5:336 (2013).
Hu et al., Proc. Natl. Acad. Sci. 108:10980 (2011).
Jia et al., Adv. Func. Mater. 22:1446 (2012).
Kaehr et al., Proc. Natl. Acad. Sci. 109.43:17336 (2012).
Klei et al., Frontiers Immunology, 8:73 (2017).
Koshkaryev et al., Advanced Drug Delivery Reviews 65:24 (2013).
Kozlovskaya et al., ACS Nano, 8:5725 (2014).
Lai & Lin, J. Cont. Rel. 134.3. 158 (2009).
Lee & Mooney, Frog. Polymer Sci. 37.1:106 (2012).
Li et al., ACS Nano 10:1317 (2016).
Liang et al., Biomaterials 35:6379 (2014).
Luk et al., Nanoscale, 6:2730 (2014).
Meli et al., Transfusion 58.2:423 (2018).
Merkel et al., Proc. Natl. Acad. Sci. 108:586 (2011).
Meyer et al., J. Am. Chem. Soc. 136:13138 (2014).
Modery-Pawlowski et al., Biomacromolecules 14:939 (2013).
Park et al., Polymers 9:140 (2017).
Rollie et al., Chemical Engineering Science 69:1 (2012).
Shang et al., J. Nanobiotechnol. 12:b67 (2014).
She et al., Advanced Materials 25:5814 (2013).
Sikora et al., Blood: blood-2014 (2014).
Su et al., Adv. Functional Mater. 26:1243 (2016).
Sun et al., ACS Macro Letters 4:1205 (2015b).
Sun et al., Life 5.1:214 (2015).
Theurl et al., Nat. Med. 22.8:945 (2016).
Townson et al., J. Am. Chem. Soc. 135:16030 (2013).
Wang et al., Chemical Science 5:3463 (2014).
Wei et al., Adv. Mater. 29.33:1701644 (2017).
Wu et al., Acs Nano 8.12:12041 (2014).
Wu et al., Nanoscale 7.32 (2015): 13680-13686.
Xu et al., Materials Today 19.9:516 (2016).
Yao et al., Small 13:1602225 (2017).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prepare a red blood cell mimetic, comprising:
a) silificating and calcinating vertebrate red blood cells, thereby providing silica replicated vertebrate red blood cells;
b) coating the silica replicated vertebrate red blood cells with one or more polyion polymers in one or more layers, thereby yielding coated silica replicates red blood cells;
c) etching the coated silica replicated red blood cells, thereby yielding red blood cell-polymer replicas; and
d) fusing the red blood cell-polymer replicas with lipid bilayers.

2. The method of claim 1 wherein prior to silification the vertebrate red blood cells are fixed.

3-4. (canceled)

5. The method of claim 1 further comprising functionalizing the desilitied, coated silica replicated red blood cells with one or more molecules.

6. The method of claim 5 wherein at least one of the molecules is a contrast agent, substrate for an enzyme, chemotherapeutic agent, nanoparticle, nucleic add, protein, peptide, antibody, drug, hemoglobin, anti-cancer agent, anti-viral agent, anti-bacterial agent, anti-parasite agent, cytokine, growth factor, or MRI contrast agent.

7.-10. (canceled)

11. The method of claim 1 wherein the silica replicated red blood cells are coated with two or more distinct polyionic polymers.

12-13. (canceled)

14. The method of claim 1 wherein the polyion comprises a polysaccharide, chitosan, polyarginine, or polylysine or comprises alginate, pectin, xanthan gum, carboxymethyl cellulose, dextran sulfate, agar, or carrageenan.

15-26. (canceled)

27. A red blood cell mimetic comprising a lipid bilayer surrounding a discoid formedof etched silica and at least one polyion layer.

28-33. (canceled)

34. The mimetic of claim 27 which comprises two or more distinct polyionic polymers.

35. The mimetic of claim 27 which comprises alternating layers of two or more distinct polyionic polymers.

36. The mimetic of claim 27 wherein one polymer is a polycationic polymer and the other polymer is a polyanionic polymer.

37. The mimetic of claim 27 wherein the polyion comprises a polysaccharide, chitosan, polyarginine, or polylysine.

38. The mimetic of claim 27 wherein the polyion comprises alginate, pectin, xanthan gum, carboxymethyl cellulose, dextran sulfate, agar, or carrageenan.

39-47. (canceled)

48. A method, comprising:
administering to a mammal a composition comprising a population of the mimetic of claim 27.

49. The method of claim 48 wherein the mammal is a human.

50. The method of claim 48 wherein the mimetic comprises one or more molecules.

51. The method of claim 48 wherein the mimetic comprises isolated protein or isolated nucleic acid or comprises an antibody or an antigen binding portion thereof.

52. (canceled)

53. The method of claim 48 wherein the lipid bilayer comprises one or more moieties.

54. The method of claim 53 wherein one of the moieties comprises antibody or antigen binding portion thereof.

55. The method of claim 48 wherein the composition is systemically administered.

56. The method of claim 48 wherein the composition is locally administered.
